SEQUENCE LISTING (12 sequences)

SEQ ID NO: 1 - DNA, 2514 bp, synthetic construct
  source 1..2514: mol_type = other DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactga

SEQ ID NO: 2 - DNA, 2511 bp, synthetic construct
  source 1..2511: mol_type = other DNA
atggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagat

SEQ ID NO: 3 - DNA, 600 bp, synthetic construct
  source 1..600: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 4 - DNA, 1624 bp, synthetic construct
  source 1..1624: mol_type = other DNA
catctcagcatcatggttggatgtttccacctggctacataagcaagctttacacaaggtgtaatttgcctaaatagtggtccattctattggggtgggagcaattgcttccaggactcacatccatatggctcccacttagccatgtggcctgctgacaaagggtggcggaactgtcactactctgttgtccacgctttcagtcctttggtttcctcttcactccctggacgctcatgtaaaaagggaggccatatacctgtgcattgtgtgtctaagcattcagtgtgtgtctaaaggcagaagggtgtgggtaggaaaacaaagacgagggaagctgcgttctccaaacacttcagacttgagtaagtggggttttgcagcaattgagtgatttgagggaaagtgaacatacaaacccaagcaatcaaagggaatattatcttaataccagggatacatgtttttctttctgcctcttaagtccaaagaggcaaatcaggacaagtggctttggttgtaaactttaaggtcaaggatcctttctgttgagcttagctctcaagttctcagtagtcaactgcggtgaaacataattaatagcacgataaatacaagttgtggaagattcgattgaaagttggaggccctctccgtggatctctctacaaagagcctgtaataaagaggacttaatcaacgttagcagggctatttaaaaagcatcgtctattaaaattcatttcttctctagagcctcttgttggagtttctctgtgtgggtgtgttcgtaagagaggaatgggttagcaagagtactgggtacaatttgtgtatccaagagaaaacagaagctctcaatgaggaagaacatatgtttctgggactgcatctgtgcaaaaagtacatagtcctgacgttgtactaagaaaaaaaacactctctttagaaagtcttttatttcacacgttatcttcttggcacatttccctcatattgccctttccgcctgaccaaatagccctttctcaccctcaggtccaggaaaaccaggaaacgtttccaacagtgcgacaaagcctgactaaccagacatactactcgctcggggatcccggaggcaagcctcagtccaagaacaggagtgactctcgagggctcacctgcctgcagggcagcccctccctgcatcgagcggaaatccatcctgtccagcgcggggcgtgggcagagcggggcgcggccccggcaggcggtatccgctgggactccgacaacgtgcgcgaccccaggcgaaccgcgcccctctccccacctccccgcgggcgggtacaagtctccaggtgtccgcgcgctcagcgggtccggcccgcccccgcccccgcccccgggcccgactgcgcgtgcccggccggagccgcgccccctcctcagggaaggccgggcgtccggcccacgaggccgagctcccccccggcccgggcctctcaccggcgcggggggcgggccaggggcggggccggactcgagcggggcggggctcgcgccagcgcccccagctccgtggcggcttcgcccgcgagtccagaggcaggcgagcagctcggtcgcccccaccggcccc

SEQ ID NO: 5 - DNA, 4324 bp, synthetic construct
  source 1..4324: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagtcatggagaagacccaccttgcagatgtcctcactggggctggcagagccggcaacctgcccaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagccaccatggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagattaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 4662 bp, synthetic construct
  source 1..4662: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaacatctcagcatcatggttggatgtttccacctggctacataagcaagctttacacaaggtgtaatttgcctaaatagtggtccattctattggggtgggagcaattgcttccaggactcacatccatatggctcccacttagccatgtggcctgctgacaaagggtggcggaactgtcactactctgttgtccacgctttcagtcctttggtttcctcttcactccctggacgctcatgtaaaaagggaggccatatacctgtgcattgtgtgtctaagcattcagtgtgtgtctaaaggcagaagggtgtgggtaggaaaacaaagacgagggaagctgcgttctccaaacacttcagacttgagtaagtggggttttgcagcaattgagtgatttgagggaaagtgaacatacaaacccaagcaatcaaagggaatattatcttaataccagggatacatgtttttctttctgcctcttaagtccaaagaggcaaatcaggacaagtggctttggttgtaaactttaaggtcaaggatcctttctgttgagcttagctctcaagttctcagtagtcaactgcggtgaaacataattaatagcacgataaatacaagttgtggaagattcgattgaaagttggaggccctctccgtggatctctctacaaagagcctgtaataaagaggacttaatcaacgttagcagggctatttaaaaagcatcgtctattaaaattcatttcttctctagagcctcttgttggagtttctctgtgtgggtgtgttcgtaagagaggaatgggttagcaagagtactgggtacaatttgtgtatccaagagaaaacagaagctctcaatgaggaagaacatatgtttctgggactgcatctgtgcaaaaagtacatagtcctgacgttgtactaagaaaaaaaacactctctttagaaagtcttttatttcacacgttatcttcttggcacatttccctcatattgccctttccgcctgaccaaatagccctttctcaccctcaggtccaggaaaaccaggaaacgtttccaacagtgcgacaaagcctgactaaccagacatactactcgctcggggatcccggaggcaagcctcagtccaagaacaggagtgactctcgagggctcacctgcctgcagggcagcccctccctgcatcgagcggaaatccatcctgtccagcgcggggcgtgggcagagcggggcgcggccccggcaggcggtatccgctgggactccgacaacgtgcgcgaccccaggcgaaccgcgcccctctccccacctccccgcgggcgggtacaagtctccaggtgtccgcgcgctcagcgggtccggcccgcccccgcccccgcccccgggcccgactgcgcgtgcccggccggagccgcgccccctcctcagggaaggccgggcgtccggcccacgaggccgagctcccccccggcccgggcctctcaccggcgcggggggcgggccaggggcggggccggactcgagcggggcggggctcgcgccagcgcccccagctccgtggcggcttcgcccgcgagtccagaggcaggcgagcagctcggtcgcccccaccggccccatggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagatgtcgacggatccggtaccgattacaaggacgacgatgacaagtgaagcttaataaaagatctttattttcattagatctgtgtgttggttttttgtgtgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 4093 bp, synthetic construct
  source 1..4093: mol_type = other DNA
acggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 8 - protein, 837 aa, synthetic construct
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 9 - DNA, 540 bp, synthetic construct
  source 1..540: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 10 - DNA, 221 bp, synthetic construct
  source 1..221: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactgggga

SEQ ID NO: 11 - DNA, 855 bp, synthetic construct
  source 1..855: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactggggactcgagttaagggcgaattcccgataaggatcttcctagagcatggctacgtagataagtagcatggcgggttaatcattaactaca

SEQ ID NO: 12 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2025/0249126 A1
(43) Pub. Date: Aug. 7, 2025

(54) PLAKOPHILLIN-2 GENE THERAPY TREATMENT METHODS

(71) Applicant: Tenaya Therapeutics, Inc., South San Francisco, CA (US)

(72) Inventors: Zhihong Jane YANG, Menlo Park, CA (US); Jin YANG, Belmont, CA (US)

(21) Appl. No.: 18/855,604

(22) PCT Filed: Apr. 10, 2023
(86) PCT No.: PCT/US2023/018082

§ 371 Date: Oct. 9, 2024

Related U.S. Application Data
(60) Provisional application No. 63/329,783, filed on Apr. 11, 2022.

(51) Int. Cl. A61K 48/00; A61K 38/17; A61P 9/00; C12N 15/86

(52) U.S. Cl. CPC A61K 48/0058; A61K 38/1709; A61K 48/0083; A61P 9/00; C12N 15/86; C12N 2750/14143; C12N 2830/008; C12N 2830/48; C12N 2830/50

(57) ABSTRACT

Provided herein are methods and compositions for plakophilin-2 gene therapy for treating heart diseases such as arrhythmogenic right ventricular cardiomyopathy (ARVC) or arrhythmogenic cardiomyopathy (ACM).

### CROSS-REFERENCE

This application claims the benefit of U.S. Provisional Application No. 63/329,783, filed Apr. 11, 2022, which is incorporated herein by reference in its entirety.

### BACKGROUND

Arrhythmogenic right ventricular cardiomyopathy (ARVC) or arrhythmogenic cardiomyopathy (ACM) is an inherited cardiac disease found in 1/2000 to 1:5000 people. ARVC is characterized by fibrofatty tissue replacement in the myocardium, myocardial atrophy, predominant right ventricular dilation, ventricular arrhythmias, and sudden cardiac death (Wang et al., 2018). The disease is difficult to diagnose by conventional imaging and ECG particularly at its early stage due to its subclinical presentations. At the late stage, the disease progresses to more overt manifestations such as ventricular arrhythmias and morphological abnormalities in the ventricle. Sudden cardiac arrest in the young and athletes is found to be associated with ARVC and exercise-related cardiac wall stress. So far, there is no effective treatment of ARVC (Wang et al., 2018).

### SUMMARY

Provided herein are methods of treating a heart disease or disorder. In some embodiments, the method comprises administering a first treatment comprising a viral vector comprising a nucleic acid encoding a plakophilin 2 (PKP2) polypeptide operatively linked to a promoter. In some embodiments. the method comprises administering a second treatment selected from the group consisting of a medication, a device, a procedure, and a lifestyle change. In some embodiments, the medication comprises one or more of an antiarrhythmic, an angiotensin-converting enzyme (ACE) inhibitor, and a beta-blocker. In some embodiments, the device comprises an implantable cardioverter-defibrillator (ICD). In some embodiments, the procedure comprises ablation. In some embodiments, the lifestyle change comprises one or more of diet, exercise, and stress reduction. In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments, the AAV9 is a variant of AAV9. In some embodiments, the heart disease or disorder is arrhythmogenic right ventricular cardiomyopathy (ARVC) or arrhythmogenic cardiomyopathy (ACM). In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enhancer. In some embodiments, the PKP2 polypeptide has an amino acid sequence of SEQ ID NO: 8. In some embodiments, the nucleic acid has a size less than or equal to about 4.7 kb. In some embodiments, the viral vector is administered in a pharmaceutically acceptable carrier or excipient comprising a buffer, a polymer, a salt, or a combination thereof. In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and/or function. In some embodiments, the method restores PKP2 mRNA expression and/or PKP2 protein and activity levels. In some embodiments, the method restores expression of one or more genes having a direct or indirect effect on one or more symptoms of the heart disease. In some embodiments, the gene comprises one or more of Ryanodine Receptor 2 (Ryr2). Ankyrin-B (Ank2), Cacnale (CaV1.2), triadin (Trdn), or calsequestrin-2 (Casq2). In some embodiments, the individual is identified as having at least one variation in a desmosome protein. In sorne embodiments, the desmosome protein is PKP2, desmoplakin (DSP), desmoglein (DSG2), desmocollin (DSC2), plakoglobin (JUP), Connexin 43 (Cx43), or transmembrane protein 43 (TMEM43). In some embodiments, the variation comprises a deletion, an insertion, a single nucleotide variation, or a copy number variation. In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14. In some embodiments, the method results in a reduction in dose or frequency of the second treatment. In some embodiments, the method results in increased exercise tolerance.

Additionally provided herein are methods of treating a heart disease or disorder in a subject having a variation in a gene encoding a protein in a desmosome. In some cases, the gene is a desmoplakin (DSP) gene, a desmoglein (DSG2) gene, a desmocollin (DSC2) gene, a plakoglobin (JUP) gene, a Connexin 43 (Cx43) gene, or a transmembrane protein 43 (TMEM43) gene. In some embodiments, the method comprises administering a first treatment comprising a viral vector comprising a nucleic acid encoding a plakophilin 2 (PKP2) polypeptide operatively linked to a promoter. In some embodiments, the variation comprises a deletion, an insertion, a single nucleotide variation, or a copy number variation. In some embodiments, the variation causes haploinsufficiency of DSP, DSG2, DSC2, JUP, Cx43, or TMEM43 protein. In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments, the AAV9 is a variant of AAV9. In some embodiments, the heart disease or disorder is arrhythmogenic right ventricular cardiomyopathy (ARVC) or arrhythmogenie cardiomyopathy (ACM). In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enhancer. In some embodiments, the PKP2 polypeptide bas an amino acid sequence of SEQ ID NO: 8. In some embodiments, the nucleic acid has a size less than or equal to about 4.7 kb. In some embodiments, the viral vector is administered in a pharmaceutically acceptable carrier or excipient comprising a buffer, a polymer, a salt, or a combination thereof. In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and or function. In some embodiments, the method restores DSP, DSG2, DSC2, JUP, Cx43, or TMEM43 protein levels. In some embodiments, the method restores expression of one or more genes having a direct or indirect effect on one or more symptoms of the heart disease. In some embodiments, the gene comprises one or more of Ryanodine Receptor 2 (Ryr2), Ankyrin-B (Ank2), Cacnalc (CaV1.2), triadin (Trdn), or calsequestrin-2 (Casq2). In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14. In some embodiments, the method results in a reduction in dose or frequency of a second treatment. In some embodiments, the method further comprises administering a second treatment selected from the group consisting of a medication, a device, a procedure, and a lifestyle change. In some embodiments, the medication comprises one or more of an antiarrhythmic, an angiotensin-converting enzyme (ACE) inhibitor, and a beta-blocker. In some embodiments, the device comprises an implantable cardioverter-defibrillator (ICD). In some embodiments, the procedure comprises ablation. In some embodiments, the lifestyle change comprises one or more of diet, exercise, and stress reduction. In some embodiments, the method results in increased exercise tolerance.

Further provided herein are methods of restoring expression of one or more genes selected from Ryanodine Receptor 2 (Ryr2), Ankyrin-B (Ank2), Caenale (CaV1.2), Triadin (Trdn), or Calsequestrin-2 (Casq2) in a subject in need thereof. In some embodiments, the method comprises administering to the subject a viral vector comprising a nucleic acid encoding a plakophilin 2 (PKP2) polypeptide operatively linked to a promoter. In some embodiments, the subject has a variation in a PKP2 gene, a desmoplakin (DSP) gene, a desmoglein (DS (2) gene, a desmocollin (DSC2) gene, a plakoglobin (JUP) gene, a Connexin 43 (Cx43) gene, or a transmembrane protein 43 (TMEM43) gene. In some embodiments, the variation comprises a deletion, an insertion, a single nucleotide variation, or a copy number variation. In some embodiments, the variation causes haploinsufficiency of DSP. DSG2, DSC2, JUP, Cx43, or TMEM43 protein. In some embodiments, the method restores DSP, DSG2, DSC2, JUP, Cx43, or TMEM43 protein levels. In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments, the AAV9 is a variant of AAV9 selected from the group consisting of CR9-10. In some embodiments, the subject has a heart disease or disorder comprising arrhythmogenie right ventricular cardiomyopathy (ARVC) or arrhythmogenic cardiomyopathy (ACM). In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enhancer. In some embodiments, the PKP2 polypeptide has an amino acid sequence of SEQ ID NO: 8. In some embodiments, the nucleic acid bas a size less than or equal to about 4.7 kb. In some embodiments, the viral vector is administered in a pharmaceutically acceptable carrier or excipient comprising a buffer, a polymer, a salt, or a combination thereof. In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and/or function. In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14. In some embodiments, the method results in a reduction in dose or frequency of a second treatment. In some embodiments, the method results in increased exercise tolerance in the subject.

Additionally provided herein are viral vectors comprising a nucleic acid encoding a plakophilin 2 (PKP2) polypeptide operatively linked to a promoter for use in methods of treating a heart disease or disorder in a subject having a variation in a gene encoding a protein in a desmosome. In some cases, the gene is a desmoplakin (DSP) gene, a desmoglein (DSG2) gene, a desmocollin (DSC2) gene, a plakoglobin (JUP) gene, a Connexin 43 (Cx43) gene, or a transmembrane protein 43 (TMEM43) gene. In some embodiments, the variation comprises a deletion, an insertion, a single nucleotide variation, or a copy number variation. In some embodiments, the variation causes haploinsufficiency of DSP, DSG2, DSC2, JUP, Cx43, or TMEM43 protein. In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAVS, and an AAV9. In some embodiments, the AAV9 is a variant of AAV9. In some embodiments, the heart disease or disorder is arrhythmogenic right ventricular cardiomyopathy (ARVC) or arrhythmogenic cardiomyopathy (ACM). In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enhancer. In some embodiments, the PKP2 polypeptide has an amino acid sequence of SEQ ID NO: 8. In some embodiments, the nucleic acid has a size less than or equal to about 4.7 kb. In some embodiments, the viral vector is administered in a pharmaceutically acceptable carrier or excipient comprising a buffer, a polymer, a salt, or a combination thereof. In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and/or function. In some embodiments, the method restores DSP, DSG2, DSC2, JUP, Cx43, or TMEM43 protein levels. In some embodiments, the method restores expression of one or more genes having a direct or indirect effect on one or more symptoms of the heart disease. In some embodiments, the gene comprises one or more of Ryanodine Receptor 2 (Ryr2), Ankyrin-B (Ank2). Caenalc (CaV1.2), triadin (Trdn), or calsequestrin-2 (Casq2). In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14. In some embodiments, the method results in a reduction in dose or frequency of a second treatment. In some embodiments, the method further comprises administering a second treatment selected from the group consisting of a medication, a device, a procedure, and a lifestyle change. In some embodiments, the medication comprises one or more of an antiarrhythmie, an angiotensin-converting enzyme (ACE) inhibitor, and a beta-blocker. In some embodiments, the device comprises an implantable cardioverter-defibrillator (ICD). In some embodiments, the procedure comprises ablation. In some embodiments, the lifestyle change comprises one or more of diet, exercise, and stress reduction. In some embodiments, the method results in increased exercise tolerance.

Further provided herein are viral vectors comprising a nucleic acid encoding a plakophilin 2 (PKP2) polypeptide operatively linked to a promoter for use in methods of restoring expression of one or more genes selected from Ryanodine Receptor 2 (Ryr2), Ankyrin-B (Ank2), Caenale (CaV1.2). Triadin (Trdn), or Calsequestrin-2 (Casq2) in a subject in need thereof. In some embodiments, the subject has a variation in a PKP2 gene, a desmoplakin (DSP) gene, a desmoglein (DSG2) gene, a desmocollin (DSC2) gene, a plakoglobin (JUP) gene, a Connexin 43 (Cx43) gene, or a transmembrane protein 43 (TMEM43) gene. In some embodiments, the variation comprises a deletion, an insertion, a single nucleotide variation, or a copy number variation. In some embodiments, the variation causes haploinsufficiency of DSP, DSG2, DSC2, JUP, Cx43, or TMEM43 protein. In some embodiments, the method restores DSP, DSG2, DSC2, JUP, Cx43, or TMEM43 protein levels. In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments, the AAV9 is a variant of AAV9 selected from the group consisting of CR9-10. In some embodiments, the subject has a heart disease or disorder comprising arrhythmogenic right ventricular cardiomyopathy (ARVC) or arrhythmogenic cardiomyopathy (ACM). In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enbancer. In some embodiments, the PKP2 polypeptide has an amino acid sequence of SEQ ID NO: 8. In some embodiments, the nucleic acid has a size less than or equal to about 4.7 kb. In some embodiments, the viral vector is administered in a pharmaceutically acceptable carrier or excipient comprising a buffer, a polymer, a salt, or a combination thereof. In some embodiments, the method reverses, reduces, or prevents at least one of fibrofatty tissue replacement; myocardial atrophy; ventricular dilation: ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and/or function. In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14. In some embodiments, the method results in a reduction in dose or frequency of a second treatment. In some embodiments, the method results in increased exercise tolerance in the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present disclosure will be obtained by reference to the following detailed description that sets forth illustrative embodiments, in which the principles of the disclosure are utilized, and the accompanying drawings of which:

FIG. 1A shows a diagram of desmosome structure in intercalated disks.

FIG. 1B shows the quantification method for colocalization of desmosome proteins, for example, the colocalization of plakoglobin (PKG) and desmoplakin (DSP). The white ‘Mask’ shows the overlap or the colocalization between the PKG and DSP.

FIG. 2 shows cell survival of cells treated with siRNA knocking down PKP2 and DSP with and without PKP2 AAV treatment.

FIG. 3A shows total PKP2 intensity in cells treated with siRNA to knockdown PKP2 or DSP with and without PKP2 AAV treatment.

FIG. 3B shows total DSP intensity in cells treated with siRNA to knockdown PKP2 or DSP with and without PKP2 AAV treatment.

FIG. 4A shows total PKP2 intensity in mask, quantifying PKP2 colocalized with DSP, in cells treated with siRNA to knockdown PKP2 or DSP with and without PKP2 AAV treatment.

FIG. 4B shows DSP signal colocalized with PKP2 signal in cells treated with siRNA to knockdown PKP2 or DSP with and without PKP2 AAV treatment.

FIG. 5A shows a Western blot showing expression PKP2 and DSP in supernatant and pellet fractions after siRNA treatment to knockdown PKP2 or DSP with and without PKP2 AAV treatment.

FIG. 5B shows quantifications of protein levels of PKP2 and DSP in the cells treated in FIG. 5A.

### DETAILED DESCRIPTION

Provided herein are methods and compositions for treating heart disease or disorder in a subject comprising administering a gene therapy construct comprising a nucleic acid encoding a plakophilin 2 (PKP2) polypeptide. In aspects, the gene therapy construct comprises an AAV capsid, for example an AAV9 capsid or a variant thereof. In additional aspects, the subject has a heart disease or disorder caused by a mutation in a desmosome gene, such as a desmoplakin (DSP) gene, desmoglein (DSG2) gene, a desmocollin (DSC2) gene, a plakoglobin (JUP) gene, a Connexin 43 (Cx43) gene, or a transmembrane protein 43 (TMEM43) gene. In further aspects, the gene therapy construct is administered in combination with additional therapies or treatments for treating the heart disease.

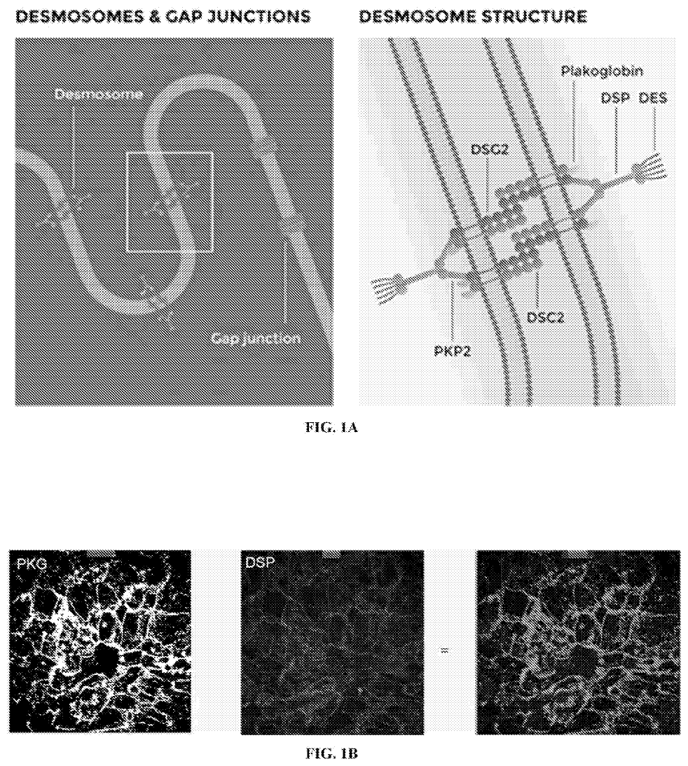

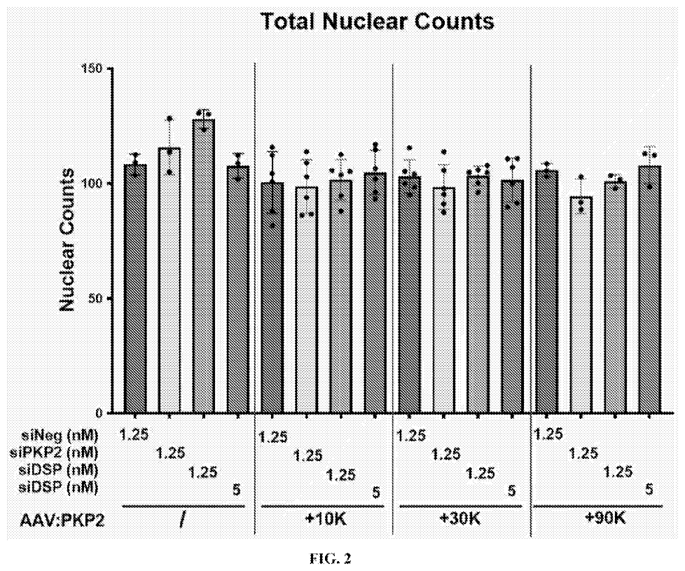

Desmosomes are specialized adhesive protein complexes that localize to intercellular junctions and maintain mechanical integrity of tissues. In some cases, they are a cell-structure specialized for cell-to-cell adhesion. In some cases, desmosomes are a strong cell-to-cell adhesion found in tissues that experience intense mechanical stress such as cardiac muscle tissue. FIG. 1A shows a simplified diagram of a desmosome and gap junction. FIG. 1B shows a mask generated based on the immunofluorescence signal of a desmosome protein, plakoglobin (PKG, left panel). The mask is generated based on a manual setting of a signal threshold to ensure a good ratio between noise and true signal. The center panel shows DSP immunofluorescence signal and the right panel shows the signal overlap between PKG and DSP.

### Methods of Treatment

Provided herein are methods of treating a heart disease or disorder in a subject. In some embodiments, the method comprises administering a treatment comprising a viral vector comprising a nucleic acid encoding a plakophilin 2 (PKP2) polypeptide operatively linked to a promoter. In some embodiments, the subject has a variation in a gene encoding a component of the desmosome. In some cases, the gene is a desmoplakin (DSP) gene, desmoglein (DSG2) gene, a desmocollin (DSC2) gene, a plakoglobin (JUP) gene, a Connexin 43 (Cx43) gene, or a transmembrane protein 43 (TMEM43) gene. In some embodiments, the variation comprises a deletion, an insertion, a single nucleotide variation, or a copy number variation. In some embodiments, the variation causes haploinsufficiency of DSP, DSG2,DSC2, JUP, Cx43, or TMEM43 protein. In some embodiments, the heart disease or disorder is arrhythmogenic right ventricular cardiomyopathy (ARVC) or arrhythmogenic cardiomyopathy (ACM).

In another aspect, provided herein are viral vectors comprising a nucleic acid encoding a plakophilin 2 (PKP2) polypeptide operatively linked to a promoter for use in treating a heart disease of disorder in a subject having a variation in a gene encoding a component of the desmosome. In some cases, the gene is a desmoplakin (DSP) gene, desmoglein (DSG2) gene, a desmocollin (DSC2) gene, a plakoglobin (JUP) gene, a Connexin 43 (Cx43) gene, or a transmembrane protein 43 (TMEM43) gene. In some embodiments, the variation comprises a deletion, an insertion, a single nucleotide variation, or a copy number variation. In some embodiments, the variation causes haploinsufficiency of DSP, DSG2,DSC2, JUP, Cx43, or TMEM43 protein. In some embodiments, the heart disease or disorder is arrhythmogenic right ventricular cardiomyopathy (ARVC) or arrhythmogenic cardiomyopathy (ACM).

In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments, the AAV9 is a variant of AAV9, such as CR9-10.

In some embodiments, the gene therapy vector comprises a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprising a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enhancer.

In some embodiments, the PKP2 polypeptide bas an amino acid sequence of SEQ ID NO: 8.

In some embodiments, the nucleic acid has a size less than or equal to about 4.7 kb.

In some embodiments, the viral vector is administered in a pharmaceutically acceptable carrier or excipient comprising a buffer, a polymer, a salt, or a combination thereof.

In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and/or function. In some embodiments, the method restores DSP, DSG2, DSC2, JUP, Cx43, or TMEM43 protein levels. In some embodiments, the method restores expression of one or more genes having a direct or indirect effect on one or more symptoms of the heart disease including but not limited to Ryanodine Receptor 2 (Ryr2), Ankyrin-B (Ank2), Caenale (CaV1.2), triadin (Trdo), or calsequestrin-2 (Casq2). In some embodiments, the method results in increased exercise tolerance.

In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14. In some embodiments, the method results in a reduction in dose or frequency of a second treatment.

### Combination Methods of Treatment

Also provided herein are methods of treating a heart disease or disorder comprising: administering a first treatment comprising a viral vector comprising a nucleic acid encoding a plakophilin 2 (PKP2) polypeptide operatively linked to a promoter and administering a second treatment selected from the group consisting of a medication, a device, a procedure, and a lifestyle change. In some embodiments, the medication comprises one or more of an antiarrhythmic, an angiotensin-converting enzyme (ACE) inhibitor, and a beta-blocker. In some embodiments, the device comprises an implantable cardioverter-defibrillator (ICD). In some embodiments, the procedure comprises ablation. In some embodiments, the lifestyle change comprises one or more of diet, exercise, and stress reduction. In some embodiments, the method results in a reduction in dose or frequency of the second treatment. In some embodiments, the method results in increased exercise tolerance.

In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments. the AAV9 is a variant of AAV9, such as CR9-10.

In some embodiments, the heart disease or disorder is arrhythmogenic right ventricular cardiomyopathy (ARVC) or arrhythmogenic cardiomyopathy (ACM).

In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodiments, the viral vector further comprises a cardiac specific enhancer. In some embodiments, the PKP2 polypeptide has an amino acid sequence of SEQ ID NO: 8.

In some embodiments, the nucleic acid has a size less than or equal to about 4.7 kb.

In some embodiments, the viral vector is administered in a pharmaceutically acceptable carrier or excipient comprising a buffer, a polymer, a salt, or a combination thereof.

In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method restores desmosome structure and/or function. In some embodiments, the method restores PKP2 mRNA expression and/or PKP2 protein and activity levels. In some embodiments, the method restores expression of one or more genes having a direct or indirect effect on one or more symptoms of the heart disease. In some embodiments, the gene comprises one or more of Ryanodine Receptor 2 (Ryt2), Ankyrin-B (Ank2), Cacnale (CaV1.2), triadin (Trdn), or calsequestrin-2 (Casq2).

In some embodiments, the individual is identified as having at least one variation in a desmosome protein. In some embodiments, the desmosome protein is PKP2, desmoplakin (DSP), desmoglein (DSG2), desmocollin (DSC2), plakoglobin (JUP), a Connexin 43 (Cx43) gene, or transmembrane protein 43 (TMEM43). In some embodiments, the variation comprises a deletion, an insertion, a single nucleotide variation, or a copy number variation.

In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14.

### Methods of Restoring Gene Expression

In another aspect, there are provided methods of restoring expression of one or more genes selected from Ryanodine Receptor 2 (Ryt2), Ankyrin-B (Ank2), Cacnale (CaV1.2), Triadin (Trdn), or Calsequestrin-2 (Casq2) in a subject in need thereof, comprising administering to the subject a viral vector comprising a nucleic acid encoding a plakophilin 2 (PKP2) polypeptide operatively linked to a promoter.

In another aspect, provided herein are viral vectors comprising a nucleic acid encoding a PKP2 gene operatively linked to a promoter for use in restoring expression of one or more genes selected from Ryanodine Receptor 2 (Ryr2), Ankyrin-B (Ank2), Cacnale (CaV1.2), Triadin (Trdn), or Calsequestrin-2 (Casq2) in a subject in need thereof.

In some embodiments, the subject has a variation in a PKP2 gene, a desmoplakin (DSP) gene, desmoglein (DSG2) gene, a desmocollin (DSC2) gene, a plakoglobin (JUP) gene, a Connexin 43 (Cx43) gene, or a transmembrane protein 43 (TMEM43) gene. In some embodiments, the variation comprises a deletion, an insertion, a single nucleotide variation, or a copy number variation. In some embodiments, the variation causes haploinsufficiency of DSP, DSG2, DSC2, JUP, Cx43, or TMEM43 protein. In some embodiments, the method restores DSP, DSG2, DSC2, JUP, Cx43, or TMEM43 protein levels.

In some embodiments, the viral vector is selected from the group consisting of an adeno-associated virus, an adenovirus, a lentivirus, a pox virus, a vaccinia virus, or a herpes virus. In some embodiments, the viral vector is an adeno-associated virus. In some embodiments, the adeno-associated virus is selected from the group consisting of an AAV6, an AAV8, and an AAV9. In some embodiments, the AAV9 is a variant of AAV9, such as CR9-10.

In some embodiments, the subject has a heart disease or disorder is arrhythmogenic right ventricular cardiomyopathy (ARVC) or arrhythmogenic cardiomyopathy (ACM).

In some embodiments, the promoter is a promoter that causes expression in tissues including the heart or a cardiac specific promoter. In some embodiments, the cardiac specific promoter is a PKP2 promoter, a troponin promoter, or an alpha-myosin heavy chain promoter. In some embodiments, the viral vector comprises a 3′ element comprises a Woodchuck Hepatitis Virus Posttransciptional Regulatory Element (WPRE), a bovine growth hormone polyadenylation (bGH polyA) sequence, or a combination thereof. In some embodimentis, the viral vector further comprises a cardiac specific enhancer.

In some embodiments, the PKP2 polypeptide has an amino acid sequence of SEQ ID NO: 8.

In some embodiments, the nucleic acid has a size less than or equal to about 4.7 kb.

In some embodiments, the viral vector is administered in a pharmaceutically acceptable carrier or excipient comprising a buffer, a polymer, a salt, or a combination thereof.

In some embodiments, the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure: left ventricular cardiomyopathy, dilation, or heart failure; atrial arrhythmias; syncope; palpitations; shortness of breath; or chest pain. In some embodiments, the method restores desmosome structure and/or function.

In some embodiments, the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14.

In some embodiments, the method results in increased exercise tolerance in the subject.

### Gene Therapy Vectors

In another aspect, there are provided gene therapy vectors comprising a plakophilin 2 gene operatively linked to at least one promoter. In some cases, the gene therapy vector comprises a viral vector. In some cases, the viral vector is any suitable viral vector for treating a heart disease or condition. In some cases, the viral vector is selected from the group consisting of an adeno-associated virus, an adenovirus, a lentivirus, a pox virus, a vaccinia virus, or a herpes virus. In some cases, the gene therapy vector is an adeno-associated virus. In some cases, the adeno-associated virus is selected from the group consisting of an AAV6, an AAVS, and an AAV9, or a derivative thereof. In some cases, the adeno-associated virus is an AAV9 or a derivative thereof. In some cases, the AAV9 has a nucleic acid sequence with at least 95% identity SEQ ID NO: 7. In some cases, the adeno-associated virus is a derivative of AAV6, AAV8, or AAV9, optimized for transducing cells according to methods of treatment herein. In some embodiments, the adeno-associated virus is a variant of AAV6, AAV8, AAV9, or other adenovirus optimized for transducing cells according to methods herein. In some cases, the derivative is any AAV described in U.S. Patent Application No. 63/012,703, which is hereby incorporated by reference in its entirety.

In some embodiments of gene therapy vectors provided herein. PKP2 is expressed by any promoter suitable for expression in the affected cells and tissues, for example cardiomyocytes. For example, in some cases, the promoter is a cardiac specific promoter. In some cases, the cardiac specific promoter is a troponin promoter or an alpha-myosin heavy chain promoter. In some cases, the promoter is a PKP2 promoter. In some cases, a cardiac specific enhancer is combined with the promoter. In some cases, the troponin promoter bas a nucleic acid sequence having at least 80%, 85%, 90%, 95%, or 99% identity to SEQ ID NO: 3. In some cases, the PKP2 promoter has a nucleic acid sequence having at least 80%, 85%, 90%, 95%, or 99% identity to SEQ ID NO: 4. In some cases, the promoter is a constitutive promoter. In some cases, the constitutive promoter is a beta-actin promoter.

In some embodiments of gene therapy vectors provided herein the nucleic acid encoding the PKP2 gene bas any suitable sequence encoding a PKP2 polypeptide for example, any nucleic acid encoding a polypeptide having a sequence of SEQ ID NO: 8. For example, in some cases, the PKP2 gene has a sequence having at least 80%, 85%, 90%, 95%, or 99% identity to SEQ ID NO: 1. In some cases, the PKP2 gene has a sequence having at least 80%, 85%, 90%, 95%, or 99% identity to SEQ ID NO: 2. In some cases, the nucleic acid sequence encoding the PKP2 gene is codon optimized.

In some embodiments of gene therapy vectors provided berein, the gene therapy vector comprises a 3′ element. In some embodiments, the 3′ element stabilizes the transcriptional product of the gene therapy vector (e.g., the PKP2 transcript). In some embodiments, the 3′ element comprises a bovine growth hormone (BGH) polyadenylation sequence. In some embodiments, the 3′ element comprises a woodchuck hepatitis virus posttranscriptional regulatory element (WPRE).

In some embodiments of gene therapy vectors provided herein, the gene therapy vector has a gene expression cassette having a size of about 3 kb to about 5 kb. In some embodiments, the gene expression cassette has a size of about 4 kb to about 5 kb. In some embodiments, the gene expression cassette has a size of about 4.2 kb to about 4.8 kb. In some embodiments, the gene expression cassette has a size of about 4.5 kb. In some embodiments, the gene expression cassette has a size no larger than about 5 kb. In some embodiments, the gene expression cassette bas a size no larger than about 4.9 kb. In some embodiments, the gene expression cassette has a size no larger than about 4.8 kb. In some embodiments. the gene expression cassette has a size no larger than about 4.7 kb. In some embodiments, the gene expression cassette has a size no larger than about 4.6 kb. In some embodiments, the gene expression cassette has a size no larger than about 4.5 kb. In some embodiments, the gene expression cassette bas a size no larger than about 4.4 kb. In some embodiments, the gene expression cassette has a size no larger than about 4.3 kb. In some embodiments, the gene expression cassette has a size no larger than about 4.2 kb. In some embodiments, the gene expression cassette has a size no larger than about 4.1 kb. In some embodiments, the gene expression cassette has a size no larger than about 4 kb. In some embodiments, the gene expression cassette has a size no larger than about 3.9 kb. In some embodiments, the gene expression cassette has a size no larger than about 3.8 kb. In some embodiments, the gene expression cassette has a size no larger than about 3.7 kb. In some embodiments, the gene expression cassette has a size no larger than about 3.6 kb. In some embodiments, the gene expression cassette has a size no larger than about 3.5 kb. In some embodiments, the gene expression cassette has a size of at least about 3.1 kb. In some embodiments, the gene expression cassette has a size of at least about 3.3 kb. In some embodiments, the gene expression cassette has a size of at least about 3.5 kb. In some embodiments, the gene expression cassette has a size of at least about 3.7 kb. In some embodiments, the gene expression cassette has a size of at least about 3.9 kb. In some embodiments, the gene expression cassette has a size of at least about 4.1 kb. In some embodiments, the gene expression cassette has a size of at least about 4.2 kb. In some embodiments, the gene expression cassette has a size of at least about 4.3 kb. In some embodiments, the gene expression cassette has a size of at least about 4.4 kb. In some embodiments, the gene expression cassette has a size of at least about 4.5 kb. In some embodiments, the gene expression cassette has a size of at least about 4.6 kb. In some embodiments, the gene expression cassette has a size of at least about 4.7 kb. In some embodiments, the gene expression cassette has a size of at least about 4.8 kb. In some embodiments, the gene expression cassette has a size of at least about 4.9 kb. In some embodiments, the gene expression cassette bas a size of at least about 5 kb.

In various embodiments of gene therapy vectors provided herein, the gene therapy vector comprising a PKP2 gene is formulated in a composition comprising a pharmaceutically acceptable carrier or excipient. For example, in some cases, the pharmaceutically acceptable carrier or excipient comprises a buffer, a polymer, a salt, or a combination thereof.

In some embodiments, gene therapy vectors berein comprise nucleic acid sequences provided in Table 1 below.

TABLE 1
Sequences

| | | SEQ |
|---|---|---|
| | | ID |
| Name | Sequence | NO: |
| Human | ATGGCAGCCCCCGGCGCCCCAGCTGAGTACGGCTACATCCGGAC | 1 |
| PKP2 | CGTCCTGGGCCAGCAGATCCTGGGACAACTGGACAGCTCCAGCC | |
| | TGGCGCTGCCCTCCGAGGCCAAGCTGAAGCTGGCGGGGAGCAGC | |
| | GGCCGCGGCGGCCAGACAGTCAAGAGCCTGCGGATCCAGGAGC | |
| | AGGTGCAGCAGACCCTCGCCCGGAAGGGCCGCAGCTCCGTGGGC | |
| | AACGGAAATCTTCACCGAACCAGCAGTGTTCCTGAGTATGTCTA | |
| | CAACCTACACTTGGTTGAAAATGATTTTGTTGGAGGCCGTTCCCC | |
| | TGTTCCTAAAACCTATGACATGCTAAAGGCTGGCACAACTGCCA | |
| | CTTATGAAGGTCGCTGGGGAAGAGGAACAGCACAGTACAGCTCC | |
| | CAGAAGTCCGTGGAAGAAAGGTCCTTGAGGCATCCTCTGAGGAG | |
| | ACTGGAGATTTCTCCTGACAGCAGGCCGGAGAGGGCTCACTACA | |
| | CGCACAGCGATTACCAGTACAGCCAGAGAAGCCAGGCTGGGCA | |
| | CACCCTGCACCACCAAGAAAGCAGGGGGGCCGCCCTCCTAGTGC | |
| | CACCGAGATATGCTCGTTCCGAGATCGTGGGGGTCAGCCGTGCT | |
| | GGCACCACAAGCAGGCAGCGCCACTTTGACACATACCACAGACA | |
| | GTACCAGCATGGCTCTGTTAGCGACACCGTTTTTGACAGCATCCC | |
| | TGCCAACCCGGCCCTGCTCACGTACCCCAGGCCAGGGACCAGCC | |
| | GCAGCATGGGCAACCTCTTGGAGAAGGAGAACTACCTGACGGC | |
| | AGGGCTCACTGTCGGGCAGGTCAGGCCGCTGGTGCCCCTGCAGC | |
| | CCGTCACTCAGAACAGGGCTTCCAGGTCCTCCTGGCATCAGAGC | |
| | TCCTTCCACAGCACCCGCACGCTGAGGGAAGCTGGGCCCAGTGT | |
| | CGCCGTGGATTCCAGCGGGAGGAGAGCGCACTTGACTGTCGGCC | |
| | AGGCGGCCGCAGGGGGAAGTGGGAATCTGCTCACTGAGAGAAG | |
| | CACTTTCACTGACTCCCAGCTGGGGAATGCAGACATGGAGATGA | |
| | CTCTGGAGCGAGCAGTGAGTATGCTCGAGGCAGACCACATGCTG | |
| | CCATCCAGGATTTCTGCTGCAGCTACTTTCATACAGCACGAGTGC | |
| | TTCCAGAAATCTGAAGCTCGGAAGAGGGTTAACCAGCTTCGTGG | |
| | CATCCTCAAGCTTCTGCAGCTCCTAAAAGTTCAGAATGAAGACG | |
| | TTCAGCGAGCTGTGTGTGGGGCCTTGAGAAACTTAGTATTTGAA | |
| | GACAATGACAACAAATTGGAGGTGGCTGAACTAAATGGGGTAC | |
| | CTCGGCTGCTCCAGGTGCTGAAGCAAACCAGAGACTTGGAGACT | |
| | AAAAAACAAATAACAGGTTTGCTGTGGAATTTGTCATCTAATGA | |
| | CAAACTCAAGAATCTCATGATAACAGAAGCATTGCTTACGCTGA | |
| | CGGAGAATATCATCATCCCCTTTTCTGGGTGGCCTGAAGGAGAC | |
| | TACCCAAAAGCAAATGGTTTGCTCGATTTTGACATATTCTACAAC | |
| | GTCACTGGATGCCTAAGAAACATGAGTTCTGCTGGCGCTGATGG | |
| | GAGAAAAGCGATGAGAAGATGTGACGGACTCATTGACTCACTG | |
| | GTCCATTATGTCAGAGGAACCATTGCAGATTACCAGCCAGATGA | |
| | CAAGGCCACGGAGAATTGTGTGTGCATTCTTCATAACCTCTCCTA | |
| | CCAGCTGGAGGCAGAGCTCCCAGAGAAATATTGCCAGAATATCT | |
| | ATATTCAAAACCGGAATATCCAGACTGACAACAACAAAAGTATT | |
| | GGATGTTTTGGCAGTCGAAGCAGGAAAGTAAAAGAGCAATACC | |
| | AGGACGTGCCGATGCCGGAGGAAAAGAGCAACCCCAAGGGCGT | |
| | GGAGTGGCTGTGGCATTCCATTGTTATAAGGATGTATCTGTGCTT | |
| | GATCGCCAAAAGTGTCCGCAACTACACACAAGAAGCATCCTTAG | |
| | GAGCTCTGCAGAACCTCACGGCCGGAAGTGGACCAATGCCGACA | |
| | TCAGTGGCTCAGACAGTTGTCCAGAAGGAAAGTGGGCTGCAGCA | |
| | CACCCGAAAGAIGCTGCATGTTGGIGACCCAAGTGTGAAAAAGA | |
| | CAGCCATCTCGCTGCTGAGGAATCTGTCCCGGAATCTTTCTCTGC | |
| | AGAATGAAATTGCCAAAGAAACTCTCCCTGATTTGGTTTCCATC | |
| | ATTCCTGACACAGTCCCGAGTACTGACCTTCTCATTGAAACTACA | |
| | GCCTCTGCCTGTTACACATTGAACAACATAATCCAAAACAGTTA | |
| | CCAGAATGCACGCGACCTTCTAAACACCGGGGGCATCCAGAAAA | |
| | TTATGGCCATTAGTGCAGGCGATGCCTATGCCTCCAACAAAGCA | |
| | AGTAAAGCTGCTTCCGTCCTTCTGTATTCTCTGTGGGCACACACG | |
| | GAACTGCATCATGCCTACAAGAAGGCTCAGTTTAAGAAGACAGA | |
| | TTTTGTCAACAGCCGGACTGCCAAAGCCTACCACTCCCTTAAAG | |
| | ACTGA | |
| Human | ATGGCTGCTCCTGGTGCTCCTGCCGAGTACGGCTACATCAGAAC | 2 |
| PKP2 | ACTGCTGGGCCAGCAGATCCTGGGACAGCTGGATTCTAGCTCTC | |
| (codon | TGGCCCTGCCTTCTGAGGCCAAGCTGAAACTGGCCGGCAGTTCT | |
| optimized) | GGAAGAGGGGGCCAGACAGTGAAGTCCCTGCGGATCCAAGAAC | |
| | AGGTGCAGCAGACCCTGGCCAGAAAGGGCAGATCTTCTGTCGGC | |
| | AACGGCAACCTGCACAGAACCAGCTCTGTGCCCGAGTACGTGTA | |
| | CAATCTGCACCTGGTGGAAAACGACTTCGTCGGCGGCAGATGCC | |
| | CTGTGCCTAAGACCTACGATATGCTGAAGGCCGGCACCACCGCC | |
| | ACCTATGAAGGCAGATGGGGAAGAGGCACAGCCCAGTACAGCA | |
| | GCCAGAAAAGCGTGGAAGAGAGAAGCCTGCGGCACCCTCTGCG | |
| | GAGACTGGAAATCAGCCCTGATAGCAGCCCAGAGAGAGCCCAC | |
| | TACACCCACAGCGACTACCAGTACTCCCAGAGATCTCAGGCCGG | |
| | CCACACACTGCACCACCAAGAGTCTAGAAGGGCCGCTCTGCTGG | |
| | TGCCTCCTAGATACGCCAGATCTGAGATCGTGGGCGTGTCCAGA | |
| | GCCGGCACAACAAGCAGACAGAGACACTTCGACACCTACCACC | |
| | GGCAGTATCAGCACGGCAGCGTGTCCGATACCGTGTTCGATAGC | |
| | ATCCCCGCCAATCCTGCTCTGCTGACATACCCTAGACCTGGCACC | |
| | TCCAGATCCATGGGCAATCTGCTGGAAAAAGAGAACTACCTGAC | |
| | CGCCGGACTGACCGTGGGACAAGTTCGACCTCTGGTTCCTCTGC | |
| | AGCCCGTGACACAGAACAGAGCCAGCAGAAGCAGCTGGCACCA | |
| | GTCCAGCTTCCACAGCACCAGAACACTGAGAGAAGCTGGCCCTA | |
| | GCGTGGCCGTGGATTCTTCTGGTAGAAGGGCTCACCTGACAGTT | |
| | GGCCAAGCAGCTGCAGGCGGAAGCGGAAATCTGCTGACCGAGA | |
| | GAAGCACCTTCACCGACAGCCAGCTGGGCAACGCCGACATGGA | |
| | AATGACACTGGAACGGGCCGTGTCCATGCTGGAAGCCGATCACA | |
| | TGCTGCCCAGCAGAATTAGCGCCGCTGCCACCTTTATCCAGCAC | |
| | GAGTGCTTCCAGAAGTCTGAGGCCCGGAAGAGAGTGAACCAGCT | |
| | GAGAGGCATCCTGAAGCTGCTGCAGCTCCTGAAGGTGCAGAACG | |
| | AGGATGTGCAGAGGGCTGTGTGTGGGGCCCTGAGAAATCTGGTG | |
| | TTCGAGGACAACGACAACAAGCTGGAAGTGGCCGAGCTGAACG | |
| | GCGTGCCAAGACTGCTGCAGGTTCTGAAACAGACCCGCGACCTG | |
| | GAAACAAAGAAGCAGATCACCGGCCTGCTCTGGAACCTGAGCA | |
| | GCAACGACAAGCTGAAGAACCTGATGATCACAGAGGCCCTGCTG | |
| | ACCCTGACAGAGAACATCATCATCCCTTTCAGCGGCTGGCCCGA | |
| | GGGCGATTACCCTAAAGCTAATGGCCTGCTGGACTTCGACATCT | |
| | TCTACAACGTGACCGGCTGCCTGAGAAACATGTCTAGCGCTGGC | |
| | GCCGATGGCAGAAAGGCCATGAGAAGATGTGACGGCCTGATCG | |
| | ACAGCCTGGTGCACTATGTGCGGGGCACAATCGCCGATTACCAG | |
| | CCTGATGATAAGGCCACCGAGAACTGCGTGTGCATGCTGCACAA | |
| | CCTGAGCTACCAGCTGGAAGCAGAGCTGCCCGAGAAGTACAGCC | |
| | AGAACATCTACATCCAGAACCGGAACATCCAGACCGACAACAA | |
| | CAAGAGCATCGGCTGCTTCGGCAGCCGCAGCCGGAAAGTGAAA | |
| | GAACAGTACCAGGACGTGCCCATGCCTGAGGAAAAGTCTAACCC | |
| | CAAAGGCGTGGAATGGCTGTGGCACAGCATCGTGATCCGGATGT | |
| | ACCTGAGCCTGATCGCCAAGAGCGTGCGGAATTACACCCAAGAG | |
| | GCATCTCTGGGCGCCCTGCAGAATCTGACAGCAGGATCTGGCCC | |
| | TATGCCTACCTCTGTGGCTCAGACCGTGGTGCAGAAAGAGTCTG | |
| | GCCTGCAGCACACCCGGAAGATGCTGCATGTGGGAGATCCCAGC | |
| | GTGAAGAAAACCGCCATCAGCCTGCTGAGAAACCTGAGCCGGA | |
| | ATCTGTCTCTGCAGAATGAGATCGCCAAAGAGACACTGCCCGAC | |
| | CTGGTGTCTATCATCCCTGACACCGTGCCTAGCACCGACCTGCTG | |
| | ATTGAGACAACAGCCAGCGCCTGCTACACCCTGAACAACATCAT | |
| | TCAGAACTCCTACCAGAACGCCCGCGATCTGCTGAACACAGGGG | |
| | GCATCCAGAAAATCATGGCCATCTCTGCCGGCGACGCCTACGCC | |
| | TCTAACAAGGCCTCTAAAGCCGCCAGCGTGCTGCTGTATTCTCTG | |
| | TGGGCCCATACCGAGCTGCACCATGCCTATAAGAAGGCCCAGTT | |
| | CAAAAAGACCGACTTCGTGAACAGCCGGACCGCCAAGGCCTACC | |
| | ACTCTCTGAAAGAT | |
| pcTNT | GTCATGGAGAAGACCCACCTTGCAGATGTCCTCACTGGGGCTGG | 3 |
| Promoter | CAGAGCCGGCAACCTGCCTAAGGCTGCTCAGTCCATTAGGAGCC | |
| | AGTAGCCTGGAAGATGTCTTTACCCCCAGCATCAGTTCAAGTGG | |
| | AGCAGCACATAACTCTTGCCCTCTGCCTTGCAAGATTCTGGTGCT | |
| | GAGACTTATGGAGTGTCTTGGAGGTTGCCTTCTGCCCCCCAACCC | |
| | TGCTCCCAGCTGGCCCTCCCAGGCCTGGGTTGCTGGCCTCTGCTT | |
| | TATCAGGATTCTCAAGAGGGACAGCTGGTTTATGTTGCATGACT | |
| | GTTCCCTGCATATCTGCTCTGGTTTTAAATAGCTTATCTGAGCAG | |
| | CTGGAGGACCACATGGGCTTATATGGCGTGGGGTACATGTTCCT | |
| | GTAGCCTTGTCCCTGGCACCTGCCAAAATAGCAGCCAACACCCC | |
| | CCACCCCCACCGCCATCCCCCTGCCCCACCCGTCCCCTGTCGCAC | |
| | ATTCCTCCCTCCGCAGGGCTGGCTCACCAGGCCCCAGCCCACAT | |
| | GCCTGCTTAAAGCCCTCTCCATCCTCTGCCTCACCCAGTCCCCGC | |
| | TGAGACTGAGCAGACGCCTCCA | |
| PKP2 | CATCTCAGCATCATGGTTGGATGTTTCCACCTGGCTACATAAGCA | 4 |
| promoter | AGCTTTACACAAGGTGTAATTTGCCTAAATAGTGGTCCATTCTAT | |
| | TGGGGTGGGAGCAATTGCTTCCAGGACTCACATCCATATGGCTC | |
| | CCACTTAGCCATGTGGCCTGCTGACAAAGGGTGGCGGAACTGTC | |
| | ACTACTCTGTTGTCCACGCTTTCAGTCCTTTGGTTTCCTCTTCACT | |
| | CCCTGGACGCTCATGTAAAAAGGGAGGCCATATACCTGTGCATT | |
| | GTGTGTCTAAGCATTCAGTGTGTGTCTAAAGGCAGAAGGGTGTG | |
| | GGTAGGAAAACAAAGACGAGGGAAGCTGCGTTCTCCAAACACT | |
| | TCAGACTTGAGTAAGTGGGGTTTTGCAGCAATTGAGTGATTTGA | |
| | GGGAAAGTGAACATACAAACCCAAGCAATCAAAGGGAATATTA | |
| | TCTTAATACCAGGGATACATGTTTTTCTTTCTGCCTCTTAAGTCC | |
| | AAAGAGGCAAATCAGGACAAGTGGCTTTGGTTGTAAACTTTAAG | |
| | GTCAAGGATCCTTTCTGTTGAGCTTAGCTCTCAAGTTCTCAGTAG | |
| | TCAACTGCGGTGAAACATAATTAATAGCACGATAAATACAAGTT | |
| | GTGGAAGATTCGATTGAAAGTTGGAGGCCCTCTCCGTGGATCTC | |
| | TCTACAAAGAGCCTGTAATAAAGAGGACTTAATCAACGTTAGCA | |
| | GGGCTATTTAAAAAGCATCGTCTATTAAAATTCATTTCTTCTCTA | |
| | GAGCCTCTTGTTGGAGTTTCTCTGTGTGGGTGTGTTCGTAAGAGA | |
| | GGAATGGGTTAGCAAGAGTACTGGGTACAATTTGTGTATCCAAG | |
| | AGAAAACAGAAGCTCTCAATGAGGAAGAACATATGTTTCTGGGA | |
| | CTGCATCTGTGCAAAAAGTACATAGTCCTGACGTTGTACTAAGA | |
| | AAAAAAACACTCTCTTTAGAAAGTCTTTTATTTCACACGTTATCT | |
| | TCTTGGCACATTTCCCTCATATTGCCCTTTCCGCCTGACCAAATA | |
| | GCCCTTTCTCACCCTCAGGTCCAGGAAAACCAGGAAACGTTTCC | |
| | AACAGTGCGACAAAGCCTGACTAACCAGACATACTACTGGCTCG | |
| | GGGATCCCGGAGGCAAGGCTCAGTCCAAGAACAGGAGTGACTCT | |
| | CGAGGGCTCACCTGCCTGCAGGGCAGCCCCTCCCTGCATCGAGC | |
| | GGAAATCCATCCTGTCCAGCGCGGGGCGTGGGCAGAGCGGGGC | |
| | GCGGCCCCGGCAGGGGGTATCCGCTGGGACTCCGACAACGTGGG | |
| | CGACCCCAGGCGAACCGCGCCCCTCTCCCCACCTCCCCGCGGGC | |
| | GGGTACAAGTCTCCAGGTGTCCGCGCGCTCAGCGGGTCCGGCCC | |
| | GCCCCCGCCCCCGCCCCCGGGCCCGACTGCGCGTGCCCGGCCGG | |
| | AGCCGCGCCCCCTCCTCAGGGAAGGCCGGGCGTCCGGCCCACGA | |
| | GGCCGAGCTCCCCCCCGGCCCGGGCCTCTCACCGGCGCGGGGGG | |
| | CGGGCCAGGGGGGGGGCCGGACTCGAGCGGGGGGGGGCTCGCG | |
| | CCAGCGCCCCCAGCTCCGTGGCGGCTTCGCCCGCGAGTGCAGAG | |
| | GCAGGCGAGCAGCTCGGTCGCCCCCACCGGCCCC | |
| AAV | ctgcgcgctcgctcgctcactgaggrcgcccgggcaaagcccgggcgtcgggcgacctttggtcg | 5 |
| Human | cccggcctcagtgagcgagcgagcgcgcagagagggagtggccaactccatcactaggggttcct | |
| PKP2a | tgtagttaatgattaacccgccatgctacttatctacgtagccatgctctaggaagatcggaatt | |
| Expression | cGCCCTTAAGTCATGGAGAAGACCCACCTTGCAGATGTGCTCACTGGGGCTGGCA | |
| Cassette | GAGCCGGCAACCTGCCCAAGGCTGCTCAGTCCATTAGGAGCCAG | |
| (pcTnT | TAGCCTGGAAGATGTCTTTACCCCCAGCATCAGTTCAAGTGGAG | |
| promoter, | CAGCACATAACTCTTGCCCTCTGCCTTCCAAGATTCTGGTGCTGA | |
| codon | GACTTATGGAGTGTCTTGGAGGTTGCCTTCTGCCCCCCAACCCTG | |
| optimized) | CTCCCAGCTGGCCCTCCCAGGCCTGGGTTGCTGGCCTCTGCTTTA | |
| | TCAGGATTCTCAAGAGGGACAGCTGGTTTATGTTGCATGACTGTT | |
| | CCCTGCATATCTGCTCTGGTTTTAAATAGCTTATCTGAGCAGCTG | |
| | GAGGACCACATGGGCTTATATGGCGTGGGGTACATGTTCCTGTA | |
| | GCCTTGTCCCTGGCACCTGCCAAAATAGCAGCCAACACCCCCCA | |
| | CCCCCACCGCCATCCCCCTGCCCCACCCGTCCCCTGTCGCACATT | |
| | CCTCCCTGCGCAGGGCTGGCTCACCAGGCCCCAGCCCACATGCC | |
| | TGCTTAAAGCCCTCTCCATCCTCTGCCTCACCCAGTCCCCGCTGA | |
| | GACTGAGCAGACGCCTCCAGCCACCATGGCTGCTCCTGGTGCTC | |
| | CTGCCGAGTACGGCTACATCAGAACAGTGCTGGGCCAGCAGATC | |
| | CTGGGACAGCTGGATTCTAGCTCTCTGGCCCTGCCTTCTGAGGCC | |
| | AAGCTGAAACTGGCCGGCAGTTCTGGAAGAGGCGGCCAGACAG | |
| | TGAAGTCCCTGCGGATCCAAGAACAGGTGCAGCAGACCCTGGCC | |
| | AGAAAGGGCAGATCTTCTGTCGGCAACGGCAACCTGCACAGAAC | |
| | CAGCTCTGTGCCCGAGTACGTGTACAATCTGCACCTGGTGGAAA | |
| | ACGACTTCGTCGGCGGCAGATCCCCTGTGCCTAAGACCTACGAT | |
| | ATGCTGAAGGCCGGCACCACCGCCACCTATGAAGGCAGATGGG | |
| | GAAGAGGCACAGCCCAGTACAGCAGCCAGAAAAGCGTGGAAGA | |
| | GAGAAGCCTGCGGCACCCTCTGCGGAGACTGGAAATCAGCCCTG | |
| | ATAGCAGCCCAGAGAGAGCCCACTACACCCACAGCGACTACCA | |
| | GTACTCCCAGAGATCTCAGGCCGGCCACACACTGCACCACCAAG | |
| | AGTCTAGAAGGGCCGCTCTGCTGGTGCCTCCTAGATACGCCAGA | |
| | TCTGAGATCGTGGGCGTGTCCAGAGCCGGCACAACAAGCAGACA | |
| | GAGACACTTCGACACCTACCACCGGCAGTATCAGCACGGCAGCG | |
| | TGTCCGATACCGTGTTCGATAGCATCCCCGCCAATCCTGCTCTGC | |
| | TGACATACCCTAGACCTGGCACCTCCAGATCCATGGGCAATCTG | |
| | CTGGAAAAAGAGAACTACCTGACCGCCGGACTGACCGTGGGAC | |
| | AAGTTCGACCTCTGGTTCCTCTGCAGCCCGTGACACAGAACAGA | |
| | GCCAGCAGAAGCAGCTGGCACCAGTCCAGCTTCCACAGCACCAG | |
| | AACACTGAGAGAAGCTGGCCCTAGCGTGGCCGTGGATTCTTCTG | |
| | GTAGAAGGGCTCACCTGACAGTTGGCCAAGCAGCTGCAGGCGG | |
| | AAGCGGAAATCTGCTGACCGAGAGAAGCACCTTCACCGACAGCC | |
| | AGCTGGGCAACGCCGACATGGAAATGACACTGGAACGGGCCGT | |
| | GTCCATGCTGGAAGCCGATCACATGCTGCCCAGCAGAATTAGCG | |
| | CCGCTGCCACCTTTATCCAGCACGAGTGCTTCCAGAAGTCTGAG | |
| | GCCCGGAAGAGAGTGAACCAGCTGAGAGGCATCCTGAAGCTGC | |
| | TGCAGCTCCTGAAGGTGCAGAACGAGGATGTGCAGAGGGCTGTG | |
| | TGTGGGGCCCTGAGAAATCTGGTGTTCGAGGACAACGACAACAA | |
| | GCTGGAAGTGGCCGAGCTGAACGGCGTGCCAAGACTGCTGCAG | |
| | GTTCTGAAACAGACCCGCGACCTGGAAACAAAGAAGCAGATCA | |
| | CCGGCCTGCTCTGGAACCTGAGCAGCAACGACAAGCTGAAGAAC | |
| | CTGATGATCACAGAGGCCCTGCTGACCCTGACAGAGAACATCAT | |
| | CATCCCTTTCAGCGGCTGGCCCGAGGGCGATTACCCTAAAGCTA | |
| | ATGGCCTGCTGGACTTCGACATCTTCTACAACGTGACCGGCTGCC | |
| | TGAGAAACATGTCTAGCGCTGGCGCCGATGGCAGAAAGGCCATG | |
| | AGAAGATGTGACGGCCTGATCGACAGCCTGGTGCACTATGTGCG | |
| | GGGCACAATCGCCGATTACCAGCCTGATGATAAGGCCACCGAGA | |
| | ACTGCGTGTGCATCCTGCACAACCTGAGCTACCAGCTGGAAGCA | |
| | GAGCTGCCCGAGAAGTACAGCCAGAACATCTACATCCAGAACCG | |
| | GAACATCCAGACCGACAACAACAAGAGCATCGGCTGCTTCGGCA | |
| | GCCGCAGCCGGAAAGTGAAAGAACAGTACCAGGACGTGCCCAT | |
| | GCCTGAGGAAAAGTCTAACCCCAAAGGCGTGGAATGGCTGTGGC | |
| | ACAGCATCGTGATCCGGATGTACCTGAGCCTGATCGCCAAGAGC | |
| | GTGCGGAATTACACCCAAGAGGCATCTCTGGGCGCCCTGCAGAA | |
| | TCTGACAGCAGGATCTGGCCCTATGCCTACCTCTGTGGCTCAGAC | |
| | CGTGGTGCAGAAAGAGTCTGGCCTGCAGCACACCCGGAAGATGC | |
| | TGCATGTGGGAGATCCCAGCGTGAAGAAAACCGCCATCAGCCTG | |
| | CTGAGAAACCTGAGCCGGAATCTGTCTCTGCAGAATGAGATCGC | |
| | CAAAGAGACACTGCCCGACCTGGTGTCTATCATCCCTGACACCG | |
| | TGCCTAGCACCGACCTGCTGATTGAGACAACAGCCAGCGCCTGC | |
| | TACACCCTGAACAACATCATTCAGAACTCCTACCAGAACGCCCG | |
| | CGATCTGCTGAACACAGGCGGCATCCAGAAAATCATGGCCATCT | |
| | CTGCCGGCGACGCCTACGCCTCTAACAAGGCCTCTAAAGCGGCC | |
| | AGCGTGCTGCTGTATTCTCTGTGGGCCCATACCGAGCTGCACCAT | |
| | GCCTATAAGAAGGCCCAGTTCAAAAAGACCGACTTCGTGAACAG | |
| | CCGGACCGCCAAGGCCTACCACTCTCTGAAAGATTAAtaagcttggatc | |
| | caatcaacctctggattacaaaatttgtgaaagattgactggtattcttaactatgttgctcctt | |
| | ttacgctatgtggatacgctgctttaatgcctttgtatcatgctattgcttcccgtatggctttc | |
| | attttctcctccttgtataaatcctggttgctgtctctttatgaggagttgtggcccgttgtcag | |
| | gcaacgtggcgtggtgtgcactgtgtttgctgacgcaacccccactggttggggcattgccacca | |
| | cctgtcagctcctttccgggactttcgctttccccctccctattgccacggcggaactcatcgcc | |
| | gcctgccttgcccgctgctggacaggggctcggctgttgggcactgacaattccgtggtgttgtc | |
| | ggggaaATCATcgtcctttccTtggctgctcgcctgtgttgccacctggattctgcgcgggacgt | |
| | ccttctgctacgtcccttcggccctcaatccagcggaccttccttcccgcggcctgctgccg | |
| | gctctgcggcctcttccgcgtcttcgagatctgcctcgactgtgccttctagttgccagccatct | |
| | gttgtttgcccctcccccgtgccttccttgaccctggaaggtgccactcccactgtcctttccta | |
| | ataaaatgaggaaattgcatcgcattgtctgagtaggtgtcattctattctggggggtggggggg | |
| | gcaggacagcaagggggaggattgggaagacaatagcaggcatgctggggaCTGGGGACTCGAGT | |
| | TAAGGGCgaattcccgataaggatcttcctagagcatggctacgtagataagtagcatggcgggt | |
| | taatcattaactacaaggaacccctagtgatggagttggccactccctctctgcgcgctcgctcg | |
| | ctcactgaggccgggcgaccaaaggtcgcccgacgcccgggctttgcccgggcggcctcagtgag | |
| | cgagcgagcgcgcag | |
| AAV | ctgcgcgctcgctcgctcactgaggccgcccgggcaaagcccgggcgtcgggcgacctttggtcg | 6 |
| Human | cccggcctcagtgagcgagcgagcgcgcagagagggagtggccaactccatcactaggggttcct | |
| PKP2a | tgtagttaatgattaacccgccatgctacttatctacgtagccatgctctaggaagatcggaatt | |
| Expression | cGCCCTTAACATCTCAGCATCATGGTTGGATGTTTCCACCTGGCTACATAAGCAAG | |
| Cassette | CTTTACACAAGGTGTAATTTGCCTAAATAGTGGTCCATTCTATTG | |
| (PKP2 | GGGTGGGAGCAATTGCTTCCAGGACTCACATCCATATGGCTCCC | |
| promoter, | ACTTAGCCATGTGGCCTGCTGACAAAGGGTGGCGGAACTGTCAC | |
| codon | TACTCTGTTGTCCACGCTTTCAGTCCTTTGGTTTCCTCTTCACTCC | |
| optimized) | CTGGACGCTCATGTAAAAAGGGAGGCCATATACCTGTGCATTGT | |
| | GTGTCTAAGCATTCAGTGTGTGTCTAAAGGCAGAAGGGTGTGGG | |
| | TAGGAAAACAAAGACGAGGGAAGCTGCGTTCTCCAAACACTTCA | |
| | GACTTGAGTAAGTGGGGTTTTGCAGCAATTGAGTGATTTGAGGG | |
| | AAAGTGAACATACAAACCCAAGCAATCAAAGGGAATATTATCTT | |
| | AATACCAGGGATACATGTTTTTCTTTCTGCCTCTTAAGTCCAAAG | |
| | AGGCAAATCAGGACAAGTGGCTTTGGTTGTAAACTTTAAGGTCA | |
| | AGGATCCTTTCTGTTGAGCTTAGCTCTCAAGTTCTCAGTAGTCAA | |
| | CTGCGGTGAAACATAATTAATAGCACGATAAATACAAGTTGTGG | |
| | AAGATTCGATTGAAAGTTGGAGGCCCTCTCCGTGGATCTCTCTAC | |
| | AAAGAGCCTGTAATAAAGAGGACTTAATCAACGTTAGCAGGGCT | |
| | ATTTAAAAAGCATCGTCTATTAAAATTCATTTCTTCTCTAGAGCC | |
| | TCTTGTTGGAGTTTCTCTGTGTGGGTGTGTTCGTAAGAGAGGAAT | |
| | GGGTTAGCAAGAGTACTGGGTACAATTTGTGTATCCAAGAGAAA | |
| | ACAGAAGCTCTCAATGAGGAAGAACATATGTTTCTGGGACTGCA | |
| | TCTGTGCAAAAAGTACATAGTCCTGACGTTGTACTAAGAAAAAA | |
| | AACACTCTCTTTAGAAAGTCTTTTATTTCACACGTTATCTTCTTGG | |
| | CACATTTCCCTCATATTGCCCTTTCCGCCTGACCAAATAGCCCTT | |
| | TCTCACCCTCAGGTCCAGGAAAACCAGGAAACGTTTCCAACAGT | |
| | GCGACAAAGCCTGACTAACCAGACATACTACTCGCTCGGGGATC | |
| | CCGGAGGCAAGCCTCAGTCCAAGAACAGGAGTGACTCTCGAGG | |
| | GCTCACCTGCCTGCAGGGCAGCCCCTCCCTGCATCGAGCGGAAA | |
| | TCCATCCTGTCCAGCGCGGGGCGTGGGCAGAGCGGGGCGCGGCC | |
| | CCGGCAGGGGGTATCCGCTGGGACTCCGACAACGTGCGCGACCC | |
| | CAGGCGAACCGCGCCCCTCTCCCCACCTCCCCGGGGGCGGGTAC | |
| | AAGTCTCCAGGTGTCCGCGCGCTCAGCGGGTCCGGCCCGCCCCC | |
| | GCCCCCGCCCCCGGGCCCGACTGCGCGTGCCCGGCCGGAGCCGC | |
| | GCCCCCTCCTCAGGGAAGGCCGGGCGTCCGGCCCACGAGGCCGA | |
| | GCTCCCCCCCGGCCCGGGCCTCTCACCGGCGCGGGGGGCGGGCC | |
| | AGGGGGGGGGCCGGACTCGAGCGGGGCGGGGCTCGCGCCAGCG | |
| | CCCCCAGCTCCGTGGCGGCTTCGCCCGCGAGTCCAGAGGCAGGC | |
| | GAGCAGCTCGGTCGCCCCCACCGGCCCCATGGCTGCTCCTGGTG | |
| | CTCCTGCCGAGTACGGCTACATCAGAACAGTGCTGGGCCAGCAG | |
| | ATCCTGGGACAGCTGGATTCTAGCTCTCTGGCCCTGCCTTCTGAG | |
| | GCCAAGCTGAAACTGGCCGGCAGTTCTGGAAGAGGCGGCCAGA | |
| | CAGTGAAGTCCCTGCGGATCCAAGAACAGGTGCAGCAGACCCTG | |
| | GCCAGAAAGGGCAGATCTTCTGTCGGCAACGGCAACCTGCACAG | |
| | AACCAGCTCTGTGCCCGAGTACGTGTACAATCTGCACCTGGTGG | |
| | AAAACGACTTCGTCGGCGGCAGATCCCCTGTGCCTAAGACCTAC | |
| | GATATGCTGAAGGCCGGCACCACCGCCACCTATGAAGGCAGATG | |
| | GGGAAGAGGCACAGCCCAGTACAGCAGCCAGAAAAGCGTGGAA | |
| | GAGAGAAGCCTGCGGCACCCTCTGCGGAGACTGGAAATCAGCCC | |
| | TGATAGCAGCCCAGAGAGAGCCCACTACACCCACAGCGACTACC | |
| | AGTACTCCCAGAGATCTCAGGCCGGCCACACACTGCACCACCAA | |
| | GAGTCTAGAAGGGCCGCTCTGCTGGTGCCTCCTAGATACGCCAG | |
| | ATCTGAGATCGTGGGCGTGTCCAGAGCCGGCACAACAAGCAGAC | |
| | AGAGACACTTCGACACCTACCACCGGCAGTATCAGCACGGCAGC | |
| | GTGTCCGATACCGTGTTCGATAGCATCCCCGCCAATCCTGCTCTG | |
| | CTGACATACCCTAGACCTGGCACCTCCAGATCCATGGGCAATCT | |
| | GCTGGAAAAAGAGAACTACCTGACCGCCGGACTGACCGTGGGA | |
| | CAAGTTCGACCTCTGGTTCCTCTGCAGCCCGTGACACAGAACAG | |
| | AGCCAGCAGAAGCAGCTGGCACCAGTCCAGCTTCCACAGCACCA | |
| | GAACACTGAGAGAAGCTGGCCCTAGCGTGGCCGTGGATTCTTCT | |
| | GGTAGAAGGGCTCACCTGACAGTTGGCCAAGCAGCTGCAGGCG | |
| | GAAGCGGAAATCTGCTGACCGAGAGAAGCACCTTCACCGACAG | |
| | CCAGCTGGGCAACGCCGACATGGAAATGACACTGGAACGGGCC | |
| | GTGTCCATGCTGGAAGCCGATCACATGCTGCCCAGCAGAATTAG | |
| | CGCCGCTGCCACCTTTATCCAGCACGAGTGCTTCCAGAAGTCTG | |
| | AGGCCCGGAAGAGAGTGAACCAGCTGAGAGGCATCCTGAAGCT | |
| | GCTGCAGCTCCTGAAGGTGCAGAACGAGGATGTGCAGAGGGCT | |
| | GTGTGTGGGGCCCTGAGAAATCTGGTGTTCGAGGACAACGACAA | |
| | CAAGCTGGAAGTGGCCGAGCTGAACGGCGTGCCAAGACTGCTGC | |
| | AGGTTCTGAAACAGACCCGCGACCTGGAAACAAAGAAGCAGAT | |
| | CACCGGCCTGCTCTGGAACCTGAGCAGCAACGACAAGCTGAAGA | |
| | ACCTGATGATCACAGAGGCCCTGCTGACCCTGACAGAGAACATC | |
| | ATCATCCCTTTCAGCGGCTGGCCGGAGGGCGATTACCCTAAAGC | |
| | TAATGGCCTGCTGGACTTCGACATCTTCTACAACGTGACCGGCTG | |
| | CCTGAGAAACATGTCTAGCGCTGGCGCCGATGGCAGAAAGGCCA | |
| | TGAGAAGATGTGACGGCCTGATCGACAGCCTGGTGCACTATGTG | |
| | CGGGGCACAATCGCCGATTACCAGCCTGATGATAAGGCCACCGA | |
| | GAACTGCGTGTGCATCCTGCACAACCTGAGCTACCAGCTGGAAG | |
| | CAGAGCTGCCCGAGAAGTACAGCCAGAACATCTACATCCAGAAC | |
| | CGGAACATCCAGACCGACAACAACAAGAGCATCGGCTGCTTCGG | |
| | CAGCCGCAGCCGGAAAGTGAAAGAACAGTACCAGGACGTGCCC | |
| | ATGCCTGAGGAAAAGTCTAACCCCAAAGGCGTGGAATGGCTGTG | |
| | GCACAGCATCGTGATCCGGATGTACCTGAGCCTGATCGCCAAGA | |
| | GCGTGCGGAATTACACCCAAGAGGCATCTCTGGGCGCCCTGCAG | |
| | AATCTGACAGCAGGATCTGGCCCTATGCCTACCTCTGTGGCTCA | |
| | GACCGTGGTGCAGAAAGAGTCTGGCCTGCAGCACACCCGGAAG | |
| | ATGCTGCATGTGGGAGATCCCAGCGTGAAGAAAACCGCCATCAG | |
| | CCTGCTGAGAAACCTGAGCCGGAATCTGTCTCTGCAGAATGAGA | |
| | TCGCCAAAGAGACACTGCCCGACCTGGTGTCTATCATCCCTGAC | |
| | ACCGTGCCTAGCACCGACCTGCTGATTGAGACAACAGCCAGCGC | |
| | CTGCTACACCCTGAACAACATCATTCAGAACTCCTACCAGAACG | |
| | CCCCCGATCTGCTGAACACAGGCGGCATCCAGAAAATCATGGCC | |
| | ATCTCTGCCGGCGACGCCTACGCCTCTAACAAGGCCTCTAAAGC | |
| | CGCCAGCGTGCTGCTGTATTCTCTGTGGGCCCATACCGAGCTGCA | |
| | CCATGCCTATAAGAAGGCCCAGTTCAAAAAGACCGACTTCGTGA | |
| | ACAGCCGGACCGCCAAGGCCTACCACTCTCTGAAAGATGTCGAC | |
| | GGATCCGGTACCGATTACAAGGACGACGATGACAAGTGAAGCTT | |
| | AATAAAAGATCTTTATTTTCATTAGATCTGTGTGTTGGTTTTTTGT | |
| | GTGCTGGGGACTCGAGTTAAGGGCgaattcccgataaggatcttcctagagcatggc | |
| | tacgtagataagtagcatgggggttaatcattaactacaaggaacccctagtgatggagttggcc | |
| | actccctctctgcgcgctcgctcgctcactgaggccgggcgaccaaaggtcgcccgacgcccggg | |
| | ctttgcccgggcggcctcagtgagcgagcgagcgcgcag | |
| AAV9 | ACGGCGGGGTTTTACGAGATTGTGATTAAGGTCCCCAGCGACCT | 7 |
| genome | TGACGAGCATCTGCCCGGCATTTCTGACAGCTTTGTGAACTGGGT | |
| sequence | GGCCGAGAAGGAATGGGAGTTGCCGCCAGATTCTGACATGGATC | |
| | TGAATCTGATTGAGCAGGCACCCCTGACCGTGGCCGAGAAGCTG | |
| | CAGCGCGACTTTCTGACGGAATGGCGCCGTGTGAGTAAGGCCCC | |
| | GGAGGCCCTTTTCTTTGTGCAATTTGAGAAGGGAGAGAGCTACT | |
| | TCCACATGCACGTGCTCGTGGAAACCACCGGGGTGAAATCCATG | |
| | GTTTTGGGACGTTTCCTGAGTCAGATTCGCGAAAAACTGATTCA | |
| | GAGAATTTACCGCGGGATCGAGCCGACTTTGCCAAACTGGTTCG | |
| | CGGTCACAAAGACCAGAAATGGCGCCGGAGGCGGGAACAAGGT | |
| | GGTGGATGAGTGCTACATCCCCAATTACTTGCTCCCCAAAACCC | |
| | AGCCTGAGCTCCAGTGGGCGTGGACTAATATGGAACAGTATTTA | |
| | AGCGCCTGTTTGAATCTCACGGAGCGTAAACGGTTGGTGGGGCA | |
| | GCATCTGACGCACGTGTCGCAGACGCAGGAGCAGAACAAAGAG | |
| | AATCAGAATGCCAATTCTGATGCGCCGGTGATCAGATCAAAAAC | |
| | TTCAGCCAGGTACATGGAGCTGGTCGGGTGGCTCGTGGACAAGG | |
| | GGATTACCTCGGAGAAGCAGTGGATCCAGGAGGACCAGGCCTC | |
| | ATACATCTCCTTCAATGCGGCCTCCAACTCGGGGTCCCAAATCAA | |
| | GGCTGCCTTGGACAATGCGGGAAAGATTATGAGCCTGACTAAAA | |
| | CCGCCCCCGACTACCTGGTGGGCCAGCAGCCCGTGGAGGACATT | |
| | TCCAGCAATCGGATTTATAAAATTTTGGAACTAAACGGGTACGA | |
| | TCCCCAATATGCGGCTTCCGTCTTTCTGGGATGGGCCACGAAAA | |
| | AGTTCGGCAAGAGGAACACCATCTGGCTGTTTGGGCCTGCAACT | |
| | ACCGGGAAGACCAACATCGCGGAGGCCATAGCCCACACTGTGCC | |
| | CTTCTACGGGTGCGTAAACTGGACCAATGAGAACTTTCCCTTCA | |
| | ACGACTGTGTCGACAAGATGGTGATCTGGTGGGAGGAGGGGAA | |
| | GATGACCGCCAAGGTCGTGGAGTCGGCCAAAGCCATTCTCGGAG | |
| | GAAGCAAGGTGCGCGTGGACCAGAAATGCAAGTCCTCGGCCCA | |
| | GATAGACCCGACTCCCGTGATCGTCACCTCCAACACCAACATGT | |
| | GCGCCGTGATTGACGGGAACTCAACGACCTTCGAACACCAGCAG | |
| | CCGTTGCAAGACGGGATGTTCAAATTTGAACTCACCCGCCGTCT | |
| | GGATCATGACTTTGGGAAGGTCACCAAGCAGGAAGTCAAAGACT | |
| | TTTTCCGGTGGGCAAAGGATCACGTGGTTGAGGTGGAGCATGAA | |
| | TTCTACGTCAAAAAGGGTGGAGCCAAGAAAAGACCCGCCCCCA | |
| | GTGACGCAGATATAAGTGAGCCCAAACGGGTGCGCGAGTCAGTT | |
| | GCGCAGCCATCGACGTCAGACGCGGAAGCTTCGATCAACTACGC | |
| | AGACAGGTACCAAAACAAATGTTCTCGTCACGTGGGCATGAATC | |
| | TGATGCTGTTTCCCTGCAGACAATGCGAGAGAATGAATCAGAAT | |
| | TCAAATATCTGCTTCACTCACGGACAGAAAGACTGTTTAGAGTG | |
| | CTTTCCCGTGTCAGAATCTCAACCCGTTTCTGTCGTCAAAAAGGC | |
| | GTATCAGAAACTGTGCTACATTCATCATATCATGGGAAAGGTGC | |
| | CAGACGCTTGCACTGCCTGCGATCTGGTCAATGTGGATTTGGAT | |
| | GACTGCATCTTTGAACAATAAatgacttaaaccaggTATGGCTGCCGATG | |
| | GTTATCTTCCAGATTGGCTCGAGGACAACCTTAGTGAAGGAATT | |
| | CGCGAGTGGTGGGCTTTGAAACCTGGAGCCCCTCAACCCAAGGC | |
| | AAATCAACAACATCAAGACAACGCTCGAGGTCTTGTGCTTCCGG | |
| | GTTACAAATACCTTGGACCCGGCAACGGACTCGACAAGGGGGA | |
| | GCCGGTCAACGCAGCAGACGCGGCGGCCCTCGAGCACGACAAG | |
| | GCCTACGACCAGCAGCTCAAGGCCGGAGACAACCCGTACCTCAA | |
| | GTACAACCACGCCGACGCCGAGTTCCAGGAGCGGCTCAAAGAA | |
| | GATACGTCTTTTGGGGGCAACCTCGGGCGAGCAGTCTTCCAGGC | |
| | CAAAAAGAGGCTTCTTGAACCTCTTGGTCTGGTTGAGGAAGCGG | |
| | CTAAGACGGCTCCTGGAAAGAAGAGGCCTGTAGAGCAGTCTCCT | |
| | CAGGAACCGGACTCCTCCGCGGGTATTGGCAAATCGGGTGCACA | |
| | GCCCGCTAAAAAGAGACTCAATTTCGGTCAGACTGGCGACACAG | |
| | AGTCAGTCCCAGACCCTCAACCAATCGGAGAACCTCCCGCAGCC | |
| | CCCTCAGGTGTGGGATCTCTTACAATGGCTTCAGGTGGTGGCGC | |
| | ACCAGTGGCAGACAATAACGAAGGTGCCGATGGAGTGGGTAGT | |
| | TCCTCGGGAAATTGGCATTGCGATTCCCAATGGCTGGGGGACAG | |
| | AGTCATCACCACCAGCACCCGAACCTGGGCCCTGCCCACCTACA | |
| | ACAATCACCTCTACAAGCAAATCTCCAACAGCACATCTGGAGGA | |
| | TCTTCAAATGACAACGCCTACTTCGGCTACAGCACCCCCTGGGG | |
| | GTATTTTGACTTCAACAGATTCCACTGCCACTTCTCACCACGTGA | |
| | CTGGCAGCGACTCATCAACAACAACTGGGGATTCCGGCCTAAGC | |
| | GACTCAACTTCAAGCTCTTCAACATTCAGGTCAAAGAGGTTACG | |
| | GACAACAATGGAGTCAAGACCATCGCCAATAACCTTACCAGCAC | |
| | GGTCCAGGTCTTCACGGACTCAGACTATCAGCTCCCGTACGTGCT | |
| | CGGGTCGGCTCACGAGGGCTGCCTCCCGCCGTTCCCAGCGGACG | |
| | TTTTCATGATTCCTCAGTACGGGTATCTGACGCTTAATGATGGAA | |
| | GCCAGGCCGTGGGTCGTTCGTCCTTTTACTGCCTGGAATATTTCC | |
| | CGTCGCAAATGCTAAGAACGGGTAACAACTTCCAGTTCAGCTAC | |
| | GAGTTTGAGAACGTACCTTTCCATAGCAGCTACGCTCACAGCCA | |
| | AAGCCTGGACCGACTAATGAATCCACTCATCGACCAATACTTGT | |
| | ACTATCTCTCAAAGACTATTAACGGTTCTGGACAGAATCAACAA | |
| | ACGCTAAAATTCAGTGTGGCCGGACCCAGCAACATGGCTGTCCA | |
| | GGGAAGAAACTACATACCTGGACCCAGCTACCGACAACAACGT | |
| | GTCTCAACCACTGTGACTCAAAACAACAACAGCGAATTTGCTTG | |
| | GCCTGGAGCTTCTTCTTGGGCTCTCAATGGACGTAATAGCTTGAT | |
| | GAATCCTGGACCTGCTATGGCCAGCCACAAAGAAGGAGAGGAC | |
| | CGTTTCTTTCCTTTGTCTGGATCTTTAATTTTTGGCAAACAAGGA | |
| | ACTGGAAGAGACAACGTGGATGCGGACAAAGTCATGATAACCA | |
| | ACGAAGAAGAAATTAAAACTACTAACCCGGTAGCAACGGAGTC | |
| | CTATGGACAAGTGGCCACAAACCACCAGAGTGCCCAAGCACAG | |
| | GCCCAGACCGGCTGGGTTCAAAACCAAGGAATACTTCCGGGTAT | |
| | GGTTTGGCAGGACAGAGATGTGTACCTGCAAGGACGCATTTGGG | |
| | CCAAAATTCCTCACACGGACGGCAACTTTCACCCTTCTCCGCTGA | |
| | TGGGAGGGTTTGGAATGAAGCACCCGCCTCCTCAGATCCTCATC | |
| | AAAAACACACCTGTACCTGCGGATCCTCCAACGGCCTTCAACAA | |
| | GGACAAGCTGAACTCTTTCATCACCCAGTATTCTACTGGCCAAGT | |
| | CAGCGTGGAGATCGAGTGGGAGCTGCAGAAGGAAAACAGCAAG | |
| | CGCTGGAACCCGGAGATCCAGTACACTTCCAACTATTACAAGTC | |
| | TAATAATGTTGAATTTGCTGTTAATACTGAAGGTGTATATAGTGA | |
| | ACCCCGCCCCATTGGCACCAGATACCTGACTCGTAATCTGTAA | |
| PKP2 | MAAPGAPAEYGYIRTVLGQQILGQLDSSSLALPSEAKLKLAGSSGR | 8 |
| Protein | GGQTVKSLRIQEQVQQTLARKGRSSVGNGNLHRTSSVPEYVYNLH | |
| | LVENDFVGGRSPVPKTYDMLKAGTTATYEGRWGRGTAQYSSQKS | |
| | VEERSLRHPLRRLEISPDSSPERAHYTHSDYQYSQRSQAGHTLHHQE | |
| | SRRAALLVPPRYARSEIVGVSRAGTTSRQRHFDTYHRQYQHGSVSD | |
| | TVFDSIPANPALLTYPRPGTSRSMGNLLEKENYLTAGLTVGQVRPL | |
| | VPLQPVTQNRASRSSWHQSSFHSTRTLREAGPSVAVDSSGRRAHLT | |
| | VGQAAAGGSGNLLTERSTFTDSQLGNADMEMTLERAVSMLEADH | |
| | MLPSRISAAATFIQHECFQKSEARKRVNQLRGILKLLQLLKVQNEDV | |
| | QRAVCGALRNLVFEDNDNKLEVAELNGVPRLLQVLKQTRDLETKK | |
| | QITGLLWNLSSNDKLKNLMITEALLTLTENIIIPFSGWPEGDYPKAN | |
| | GLLDFDIFYNVTGCLRNMSSAGADGRKAMRRCDGLIDSLVHYVRG | |
| | TIADYQPDDKATENCVCILHNLSYQLEAELPEKYSQNIYIQNRNIQT | |
| | DNNKSIGCFGSRSRKVKEQYQDVPMPEEKSNPKGVEWLWHSIVIR | |
| | MYLSLIAKSVRNYTQEASLGALQNLTAGSGPMPTSVAQTVVQKES | |
| | GLQHTRKMLHVGDPSVKKTAISLLRNLSRNLSLQNEIAKETLPDLVS | |
| | HIPDTVPSTDLLIETTASACYTLNNIIQNSYQNARDLLNTGGIQKIMAI | |
| | SAGDAYASNKASKAASVLLYSLWAHTELHHAYKKAQFKKTDFVN | |
| | SRTAKAYHSLKD | |
| WPRE | TCAACCTCTGGATTACAAAATTTGTGAAAGATTGACTGGTATTCT | 9 |
| | TAACTATGTTGCTCCTTTTACGCTATGTGGATACGCTGCTTTAAT | |
| | GCCTTTGTATCATGCTATTGCTTCCCGTATGGCTTTCATTTTCTCC | |
| | TCCTTCTATAAATCCTGGTTGCTGTCTCTTTATGAGGAGTTGTGG | |
| | CCCGTTGTCAGGCAACGTGGCGTGGTGTGCACTGTGTTTGCTGAC | |
| | GCAACCCCCACTGGTTGGGGCATTGCCACCACCTGTCAGCTCCTT | |
| | TCCGGGACTTTCGCTTTCCCCCTCCCTATTGCCACGGCGGAACTC | |
| | ATCGCCGCCTGCCTTGCCCGCTGCTGGACAGGGGCTCGGCTGTT | |
| | GGGCACTGACAATTCCGTGGTGTTGTCGGGGAAATCATCGTGCT | |
| | TTCCTTGGCTGCTCGCCTGTGTTGCCACCTGGATTCTGCGCGGGA | |
| | CGTCCTTCTGCTACGTCCCTTCGGCCCTCAATCCAGCGGACCTTC | |
| | CTTCCCGCGGCCTGCTGCCGGCTCTGCGGCCTCTTCCGCGTCTTC | |
| | G | |
| hGH | CCTCGACTGTGCCTTCTAGTTGCCAGCCATCTGTTGTTTGCCCCT | 10 |
| polyA | CCCCCGTGCCTTCCTTGACCCTGGAAGGTGCCACTCCCACTGTCC | |
| signal | TTTCCTAATAAAATGAGGAAATTGCATCGCATTGTCTGAGTAGG | |
| | TGTCATTCTATTCTGGGGGGTGGGGTGGGGCAGGACAGCAAGGG | |
| | GGAGGATTGGGAAGACAATAGCAGGCATGCTGGGGACTGGGGA | |
| WPRE- | TCAACCTCTGGATTACAAAATTTGTGAAAGATTGACTGGTATTCT | 11 |
| hGH | TAACTATGTTGCTCCTTTTACGCTATGTGGATACGCTGCTTTAAT | |
| polyA | GCCTTTGTATCATGCTATTGCTTCCCGTATGGCTTTCATTTTCTCC | |
| signal | TCCTTGTATAAATCCTGGTTGCTGTCTCTTTATGAGGAGTTGTGG | |
| cassette | CCCGTTGTCAGGCAACGTGGCGTGGTGTGCACTGTGTTTGCTGAC | |
| | GCAACCCCCACTGGTTGGGGCATTGCCACCACCTGTCAGCTGCTT | |
| | TCCGGGACTTTCGCTTTCCCCCTCCCTATTGCCACGGCGGAACTC | |
| | ATCGCCGCCTGCCTTGCCCGCTGCTGGACAGGGGCTCGGCTGTT | |
| | GGGCACTGACAATTCCGTGGTGTTGTCGGGGAAATCATCGTCCT | |
| | TTCCTTGGCTGCTCGCCTGTGTTGCCACCTGGATTCTGCGCGGGA | |
| | CGTCCTTCTGCTACGTCCCTTCGGCCCTCAATCCAGCGGACCTTC | |
| | CTTCCCGCGGCCTGCTGCCGGCTCTGCGGCCTCTTCCGCGTCTTC | |
| | GAGATCTGCCTCGACTGTGCCTTCTAGTTGCCAGCCATCTGTTGT | |
| | TTGCCCCTCCCCCGTGCCTTCCTTGACCCTGGAAGGTGCCACTCC | |
| | CACTGTCCTTTCCTAATAAAATGAGGAAATTGCATCGCATTGTCT | |
| | GAGTAGGTGTCATTCTATTCTGGGGGGTGGGGTGGGGCAGGACA | |
| | GCAAGGGGGAGGATTGGGAAGACAATAGCAGGCATGCTGGGGA | |
| | CTGGGGACTCGAGTTAAGGGCGAATTCCCGATAAGGATCTTCCT | |
| | AGAGCATGGCTACGTAGATAAGTAGCATGGCGGGTTAATCATTA | |
| | ACTACA | |
| AAV9 | MAADGYLPDWLEDNLSEGIREWWALKPGAPQPKANQQHQDNAR | 12 |
| capsid | GLVLPGYKYLGPGNGLDKGEPVNAADAAALEHDKAYDQQLKAGD | |
| amino acid | NPYLKYNHADAEFQERLKEDTSFGGNLGRAVFQAKKRLLEPLGLV | |
| sequence | EEAAKTAPGKKRPVEQSPQEPDSSAGIGKSGAQPAKKRLNFGQTGD | |
| | TESVPDPQPIGEPPAAPSGVGSLTMASGGGAPVADNNEGADGVGSS | |
| | SGNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAHE | |
| | GCLPPFPADVFMIPQYGYLTLNDGSQAVGRSSFYCLEYFPSQMLRT | |
| | GNNFQFSYEFENVPFHSSYAHSQSLDRLMNPLIDQYLYYLSKTINGS | |
| | GQNQQTLKESVAGPSNMAVQGRNYIPGPSYRQQRVSTTVTQNNNS | |
| | EFAWPGASSWALNGRNSLMNPGPAMASHKEGEDRFFPLSGSLIFGK | |
| | QGTGRDNVDADKVMITNEEEIKTTNPVATESYGQVATNHQSAQAQ | |
| | AQTGWVQNQGILPGMVWQDRDVYLQGPIWAKIPHTDGNFHPSPL | |
| | MGGFGMKHPPPQILIKNTPVPADPPTAFNKDKLNSFITQYSTGQVSV | |
| | EIEWELQKENSKRWNPEIQYTSNYYKSNNVEFAVNTEGVYSEPRPI | |
| | GTRYLTRNL | |

### Viral Vectors

Suitable viral vectors for methods and gene therapy vectors provided herein include, but are not limited to, viral vectors (e.g. viral vectors based on vaccinia virus; poliovirus; adenovirus (e.g., Li et al. (1994) Invest Opthalmol Vis Sci 35:2543-2549; Borras et al. (1999) Gene Ther 6:515-524; Li and Davidson, (1995) Proc. Natl. Acad. Sci. 92:7700-7704; Sakamoto et al. (1999) Hum Gene Ther 5: 1088-1097; WO 94/12649; WO 93/03769; WO 93/19191; WO 94/28938; WO 95/11984 and WO 95/00655); adeno-associated virus (e.g., Ali et al. (1998) Hom Gene Ther 9 (1): 81-86, 1998, Flannery et al. (1997) Proc. Natl. Acad. Sci. 94:6916-6921; Bennett et al. (1997) Invest Opthalmol Vis Sci 38:2857-2863; Jomary et al. (1997) Gene Ther 4:683-690; Rolling et al. (1999), Hum Gene Ther 10:641-648; Ali et al. (1996) Hum Mol Genet. 5:591-594; WO 93/09239, Samulski et al. (1989) J. Vir. 63:3822-3828; Mendelson et al. (1988) Virol. 166:154-165; and Flotte et al. (1993) Proc. Natl. Acad. Sci. 90:10613-10617; SV40; herpes simplex virus; human immunodeficiency virus (e.g., Miyoshi et al. (1997) Proc. Natl. Acad. Sci. 94:10319-10323; Takahashi et al. (1999) J Virol 73:7812-7816); a retroviral vector (e.g., Marine-Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available. The following vectors are provided by way of example; for eukaryotic cells: pXT1, pSG5 (Stratagene), pSVK3, pBPV, pMSG, pSVLSV40 (Pharmacia), and pAd (Life Technologies). However, any other vector is contemplated for use so long as it is compatible with the methods of the present disclosure.

The ability of certain viruses to infect cells or enter cells via receptor-mediated endocytosis, and express viral genes stably and efficiently have made them attractive candidates for the transfer of foreign nucleic acids into cells (e.g., mammalian cells). Viral vectors are contemplated to include control sequences such as promoters for expression of the polypeptide of interest. Although many viral vectors integrate into the host cell genome, if desired, the segments that allow such integration can be removed or altered to prevent such integration. Moreover, in some embodiments, the vectors do not contain a mammalian origin of replication. Non-limiting examples of virus vectors are described below that are contemplated for use in delivering nucleic acids encoding PKP2 into a selected cell. In some embodiments, the viral vector is derived from a replication-deficient virus.

In general, other useful viral vectors are based on non-cytopathic eukaryotic viruses in which non-essential genes have been replaced with the polypeptide of interest. Non-cytopathic viruses include certain retroviruses, the life cycle of which involves reverse transcription of genomic viral RNA into DNA with subsequent proviral integration into host cellular DNA. In general, the retroviruses are replication-deficient (e.g., capable of directing synthesis of the desired transcripts, but incapable of manufacturing an infectious particle). Such genetically altered retroviral expression vectors have general utility for the high-efficiency transduction of polynucleotide in vivo.

In some embodiments, a polynucleotide encoding PKP2 is housed within an infective viras that has been engineered to express a specific binding ligand. The virus particle will thus bind with specificity to the cognate receptors of the target cell and deliver the contents to the cell. In some embodiments, the virus is modified to impart particular viral tropism, e.g., the virus preferentially infects fibroblasts, heart cells, or more particularly cardiac fibroblasts (CFs). For AAV, in some cases, capsid proteins are mutated to alter the tropism of the viral vector. For example, lentivirus tropism is often modified by using different envelope proteins; this is known as “pseudotyping.”

In some embodiments, the viral vector is a retroviral vector. Retroviruses often integrate their genes into the host genome, transfer a large amount of foreign genetic material, infect a broad spectrum of species and cell types, and are often packaged in special cell-lines (Miller et al., Am. J. Clin. Oncol, 15 (3): 216-221. 1992). In some embodiments, a retroviral vector is altered so that it does not integrate into the host cell genome.

In some embodiments, the recombinant retrovirus comprises a viral polypeptide (e.g., retroviral env) to aid entry into the target cell. Such viral polypeptides are well-established in the art, for example, U.S. Pat. No. 5,449,614. In some embodiments, the viral polypeptide is an amphotropic viral polypeptide, for example, amphotropic env, which aids entry into cells derived from multiple species. including cells outside of the original host species. In some embodiments, the viral polypeptide is a xenotropic viral polypeptide that aids entry into cells outside of the original host species. In some embodiments, the viral polypeptide is an ecotropic viral polypeptide, for example, ecotropic env, which aids entry into cells of the original host species.

Examples of viral polypeptides capable of aiding entry of retroviruses into cells include, but are not limited to: MMLV amphotropic env, MMLV ecotropic env, MMLV xenotropic env, vesicular stomatitis virus-g protein (VSV-g), HIV-1 env, Gibbon Ape Leukemia Virus (GALV) env. RD114, FeLV-C, FeLV-B, MLV 10A1 env gene, and variants thereof, including chimeras. Yee et al. (1994) Methods Cell Biol, Pt A: 99-1 12 (VSV-G); U.S. Pat. No. 5,449,614. In some cases, the viral polypeptide is genetically modified to promote expression or enhanced binding to a receptor.

In embodiments, the retroviral construct is derived from a range of retroviruses, e.g., MMLV, HIV-1, SIV, FIV, or other retrovirus described herein. In some embodiments, the retroviral construct encodes all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides often help facilitate viral entry. In an exemplary embodiment, the recombinant retrovirus is HIV-1 virus comprising a VSV-g polypeptide, but not comprising a HIV 1 env polypeptide.

In some embodiments, the retroviral construct comprises: a promoter, a multi-cloning site, and/or a resistance gene. Examples of promoters include but are not limited to CMV, SV40, EF1a, β-actin; retroviral LTR promoters, and inducible promoters. In some embodiments, the retroviral construct comprises a packaging signal (e.g., a packaging signal derived from the MFG vector; a psi packaging signal), Examples of some retroviral constructs known in the art include but are not limited to: pMX, pBabeX or derivatives thereof. Onishi et al. (1996) Experimental Hematology, 24:324-329. In some cases, the retroviral construct is a self-inactivating lentiviral vector (SIN) vector. Miyoshi et al. (1998) J. Virol 72 (10): 8150-8157. In some cases, the retroviral construct is LL-CG, LS-CG, CL-CG, CS-CG, CLG or MFG. Miyoshi et al. (1998) J. Virol 72 (10): 8150-8157; Onishi et al. (1996) Experimental Hematology, 24:324-329; Riviere et al. (1995) Proc. Natl. Acad. Sci., 92:6733-6737.

In some embodiments, a retroviral vector is constructed by inserting a nucleic acid (e.g., one encoding a polypeptide of interest or an RNA) into the viral genome in the place of some viral sequences to produce a virus that is replication-defective. To produce virions, a packaging cell line containing the gag, pol, and env genes, but without the LTR and packaging components, is constructed (Mann et al., Cell 33:153-159, 1983). When a recombinant plasmid containing a cDNA, together with the retroviral LTR and packaging sequences is introduced into a special cell line (e.g., by calcium phosphate precipitation or lipid transfection), the packaging sequence allows the RNA transcript of the recombinant plasmid to be packaged into viral particles, which are then secreted into the culture media (Nicolas and Rubinstein, In: Vectors: A survey of molecular cloning vectors and their uses, Rodriguez and Denbardt, eds., Stoneham: Butterworth, pp. 494-513. 1988; Temin. In: Gene Transfer, Kucherlapati (ed.), New York: Plenum Press, pp. 149-188, 1986; Mann et al., Cell, 33:153-159, 1983). The media containing the recombinant retroviruses is then collected, optionally concentrated, and used for gene transfer. Retroviral vectors are able to infect a broad variety of cell types. However, integration and stable expression typically involves the division of host cells (Paskind et al., Virology, 67:242-248, 1975).

In some embodiments, the viral vector is a lentiviral vector. Lentiviruses are complex retroviruses, which, in addition to the common retroviral genes gag, pol, and env, contain other genes with regulatory or structural function. Information on lentiviral vectors is available, for example, in Naldini et al., Science 272 (5259): 263-267, 1996; Zufferey et al., Nat Biotechnol 15 (9): 871-875, 1997; Blomer et al., J Virol. 71 (9): 6641-6649, 1997; U.S. Pat. Nos. 6,013,516 and 5,994,136, each of which is incorporated herein by reference in its entirety. Some examples of lentivirus include the Human Immunodeficiency Viruses: HIV-1, HIV-2 and the Simian Immunodeficiency Virus: SIV. Lentiviral vectors have been generated by attenuating the HIV virulence genes, for example, the genes env, vif, vpr, vpu and nef are deleted to make the vector biologically safe. The lentivirus employed is sometimes replication and/or integration defective.

Recombinant lentiviral vectors are capable of infecting non-dividing cells and are sometimes used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, which is incorporated herein by reference in its entirety. In some embodiments, the recombinant virus is targeted by linkage of the envelope protein with an antibody or a particular ligand for targeting to a receptor of a particular cell type. For example, a target-specific vector is sometimes generated by inserting a nucleic acid segment (including a regulatory region) of interest into the viral vector, along with another gene that encodes a ligand for a receptor on a specific target cell type.

Lentiviral vectors are known in the art, see Naldini et al., (1996 and 1998): Zufferey et al., (1997); Dull et al., 1998, U.S. Pat. Nos. 6,013,516; and 5,994,136 all incorporated herein by reference. In general, these vectors are plasmid-based or virus-based and are configured to carry the essential sequences for incorporating foreign nucleic acid, for selection and for transfer of the nucleic acid into a host cell. In some cases, a lentiviral vector is introduced into a cell concurrently with one or more lentiviral packaging plasmids, which include, without limitation, pMD2.G, pRSV-rev, pMDLG-pRRE, and pRRI-GOI. Introduction of a lentiviral vector alone or in combination with lentiviral packaging plasmids into a cell, in some embodiments causes the lentiviral vector to be packaged into a lentiviral particle. In some embodiments, the lentiviral vector is a non-integrating lentiviral (NIL) vector. Illustrative methods for generating NIL vectors, such as the D64V substitution in the integrase gene, are provided in U.S. Pat. No. 8,119,119.

In some embodiments, the viral vector is an adenoviral vector. The genetic organization of adenovirus includes an approximate 36 kb, linear, double-stranded DNA virus, which allows substitution of large pieces of adenoviral DNA with foreign sequences up to 7 kb (Grunhaus et al., Seminar in Virology 200 (2): 535-546, 1992)). In some cases, PKP2 is introduced into the cell using adenovirus assisted transfection. Increased transfection efficiencies have been reported in cell systems using adenovirus coupled systems (Kelleher and Vos, Biotechniques, 17 (6): 1110-7, 1994; Cotten et al., Proc Natl Acad Sci USA, 89 (13): 6094-6098, 1992; Curiel, Nat Immun, 13 (2-3): 141-64, 1994.).

In some embodiments, the viral vector is an adeno-associated virus (AAV) vector. AAV is an attractive vector system as it has a low frequency of integration and it can infect non-dividing cells, thus making it useful for delivery of polynucleotides into mammalian cells, for example, in tissue culture (Muzyczka, Curr Top Microbiol Immunol. 158:97-129, 1992) or in vivo. Details concerning the generation and use of rAAV vectors are described in U.S. Pat. Nos. 5,139,941 and 4,797,368, each incorporated berein by reference in its entirety.

AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and often persists essentially for the lifetime of those cells as a transcriptionally active nuclear episome (extrachromosomal element). Of particular importance to the present disclosure, AAV, and AAV9 in particular, are capable of infecting cells of the heart, such as myocardium, epicardium, or both (Prasad et al, 2011; Piras et al, 2016; Ambrosi et al., 2019). The AAV proviral genome is inserted as cloned DNA in plasmids, which makes construction of recombinant genomes feasible. Furthermore, because the signals directing AAV replication and genome encapsidation are contained within the ITRs of the AAV genome, in some cases, some or all of the internal approximately 4.3 kb of the genome (encoding replication and structural capsid proteins, rep-cap) is replaced with foreign DNA. To generate AAV vectors, in some cases, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. In some cases, AAV is even be lyophilized. Finally, AAV-infected cells are not resistant to superinfection. The AAV vectors of the disclosure include self-complementary, duplexed AAV vectors, synthetic ITRs, and/or AAV vectors with increased packaging compacity. Illustrative methods are provided in U.S. Pat. Nos. 8,784,799; 8,999,678; 9,169,494; 9,447,433; and 9,783,824, each of which is incorporated by reference in its entirety.

AAV DNA in the rAAV genomes is contemplated to be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, WO 01/83692. Other types of TAAV variants, for example rAAV with capsid mutations, are also contemplated. See, for example. Marsic et al., Mol. Therapy. 22): 1900-09 (2014). The nucleotide sequences of the genomes of various AAV serotypes are known in the art. AAV vectors of the present disclosure include AAV vectors of serotypes AAV1, AAV2, AAV4, AAV5, AAV6, AAV7, AAV8, AAV9, AAV10, AAV39, AAV43, AAV.rh74, and AAV.rh8. Illustrative AAV vectors are provided in U.S. 63/012,703: U.S. Pat. No. 7,105,345; U.S. Ser. No. 15/782,980; U.S. Pat. Nos. 7,259,151; 6,962,815; 7,718,424; 6,984,517; 7,718,424; 6,156,303; 8,524,446; 7,790,449; 7,906,111; 9,737,618; U.S. application Ser. No. 15/433,322; U.S. Pat. No. 7,198,951, each of which is incorporated by reference in its entirety.

In some embodiments, the AAV expression vector is pseudotyped to enhance targeting. To promote gene transfer and sustain expression in cardiomyocytes, AAV6, AAV8, and AAV9, are contemplated for use. In some cases, the AAV2 genome is packaged into the capsid of producing pseudotyped vectors AAV2/5, AAV2/7, and AAV2/8 respectively, as described in Balaji et al. J Surg Res. 184:691-98 (2013). In some embodiments, an AAV9 is used to target expression in myofibroblast-like lineages, as described in Piras et al. Gene Therapy 23:469-478 (2016). In some embodiments, AAV1, AAV6, or AAV9 is used, and in some embodiments, the AAV is engineered, as described in Asokari et al. Hum Gene Ther. 24:906-13 (2013); Pozsgai et al. Mol Ther. 25:855-69 (2017); Kotterman et al. Nature Reviews Genetics 15:445-51 (2014); and US20160340393A1 to Schaffer et al. In some embodiments, the viral vector is AAV engineered to increase target cell infectivity as described in US20180066285A1.

In some embodiments, the AAV vectors of the disclosure comprise a modified capsid, in particular as capsid engineered to enhance or promote in vivo or ex vivo transduction of cardiac cells, or more particularly cardiomyocytes; or that evade the subject's immune system; or that have improved biodistribution. Illustrative AAV capsids are provided in U.S. Pat. Nos. 7,867,484; No. 9,233,131; 10,046,016; WO 2016/133917; WO 2018/222503; and WO 20019/060454, each of which is incorporated by reference in its entirety. In an AAV capsid (or in particular an AAV9 capsid), one or more substitutions are contemplated to increase infectivity towards cells in the myocardium, epicardium, or both. More particularly, in some embodiments, the AAV vectors of the disclosure, optionally AAV9-based vectors, comprise in their capsid proteins one or more substitutions. In some embodiments, the AAV vectors of the disclosure comprise the AAV-A9 capsid and/or serotype. It will be appreciated that these substitutions and insertions are contemplated to be combined together to generate various capsid proteins useful in the present disclosure.

### Methods of Producing Viral Vectors

In general, a viral vector is produced by introducing a viral DNA or RNA construct into a producer cell. In some cases, the producer cell does not express exogenous genes. In other cases, the producer cell is a “packaging cell” comprising one or more exogenous genes, e.g., genes encoding one or more gag, pol, or env polypeptides and/or one or more retroviral gag, pol, or env polypeptides. In some embodiments, the retroviral packaging cell comprises a gene encoding a viral polypeptide, e.g., VSV-g, that aids entry into target cells. In some cases, the packaging cell comprises genes encoding one or more lentiviral proteins, e.g., gag, pol, env, vpr, vpu, vpx, vif, tat, rev, or nef. In some cases, the packaging cell comprises genes encoding adenovirus proteins such as E1 A or E1 B or other adenoviral proteins. For example, in some cases, proteins supplied by packaging cells are retrovirus-derived proteins such as gag. pol, and env; lentivirus-derived proteins such as gag, pol, env, vpr, vpu, vpx, vif, tat, rev, and nef; and adenovirus-derived proteins such as E1 A and E1 B. In many examples, the packaging cells supply proteins derived from a virus that differs from the virus from which the viral vector is derived. Methods of producing recombinant viruses from packaging cells and their uses are well established; see, e.g., U.S. Pat. Nos. 5,834,256; 6,910,434; 5,591,624; 5,817,491; 7,070,994; and 6,995.009.

Packaging cell lines include but are not limited to any easily-transfectable cell line. Packaging cell lines are often based on 293T cells, NIH3T3, COS or HeLa cell lines. Packaging cells are often used to package virus vector plasmids deficient in at least one gene encoding a protein required for virus packaging. Any cells that supply a protein or polypeptide lacking from the proteins encoded by such viral vectors or plasmids are contemplated for use as packaging cells. Examples of packaging cell lines include but are not limited to: Platinum-E (Plat-E), Platinum-A (Plat-A), BOSC 23 (ATCC CRL 11554) and Bing (ATCC CRL 11270). Morita et al. (2000) Gene Therapy 7 (12): 1063-1066; Onishi et al. (1996) Experimental Hematology, 24:324-329; U.S. Pat. No. 6,995,009. Commercial packaging lines are also useful, e.g., Ampho-Pak 293 cell line, Eco-Pak 2-293 cell line, RetroPack PT67 cell line, and Retro-X Universal Packaging System (all available from Clontech).

Virus vector plasmids (or constructs), include: pMXs, pMxs-IB, pMXs-puro, pMXs-neo (pMXs-IB is a vector carrying the blasticidin-resistant gene instead of the puromycin-resistant gene of pMXs-puro) Kimatura et al. (2003) Experimental Hematology 31:1007-1014; MFG Riviere et al. (1995) Proc. Natl. Acad. Sci., 92:6733-6737; pBabePuro; Morgenstern et al. (1990) Nucleic Acids Research 18:3587-3596; LL-CG, CL-CG, CS-CG, CLG Miyoshi et al. (1998) J. Vir. 72:8150-8157 and the like as the retrovirus system, and pAdexl Kanegae et al. (1995) Nucleic Acids Research 23:3816-3821 and the like as the adenovirus system. In exemplary embodiments, the retroviral construct comprises blasticidin (e.g., pMXs-IB), puromycin (e.g., pMXs-puro, pBabePuro), or neomycin (e.g., pMXs-neo). Morgenstern et al. (1990) Nucleic Acids Research 18:3587-3596.

### Promoters and Enhancers

In some embodiments, a nucleic acid encoding a PKP2 is operably linked to a promoter and/or enhancer to facilitate expression of PKP2. Depending on the host vector system utilized, any of a number of suitable transcription and translation control elements, including constitutive, tissue specific, and inducible promoters, transcription enhancer elements, transcription terminators, etc. are suitable for use in the expression vector (e.g., Bitter et al. (1987) Methods in Enzymology, 153:516-544).

Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring cardiac-specific expression will be used, including but not limited to promoters that confer expression in the myocardium, the epicardium, or both (Prasad et al., 2011). Non-limiting examples of suitable cardiac-specific promoters include alpha-myosin heavy chain (a-MHC), myosin light chain 2 (MLC-2), cardiac troponin T (cTnT), and cardiac troponin C (cTnC). In some embodiments, a PKP2 or a desmin promoter is used. In some cases, a chimeric promoter with cardiac specific expression is used. In some cases, a cardiac specific enhancer is combined with the promoter.

Examples of suitable promoters for driving expression PKP2 include, but are not limited to, retroviral long terminal repeat (I.TR) elements; constitutive promoters such as CMV, HSV1-TK, SV40, EF-1a, β-actin, phosphoglycerol kinase (PGK); inducible promoters, such as those containing Tet-operator elements; and cardiac-specific promoters, such as alpha-myosin heavy chain (a-MHC), myosin light chain 2 (MLC-2), cardiac troponin T (cTnT), and cardiac troponin C (cTnC). In some embodiments, a PKP2 or a desmin promoter is ased. In some embodiments, a chimeric promoter with cardiac specific expression is used. In some cases, a cardiac specific enhancer is combined with the promoter.

In some embodiments, a polynucleotide is operably linked to a cell type-specific transcriptional regulator element (TRE), where TREs include promoters and enhancers. Suitable TREs include, but are not limited to, TREs derived from the following genes: myosin light chain-2, α-myosin heavy chain, AE3, cardiac troponin C, and cardiac actin. Franz et al. (1997) Cardiovasc. Res. 35:560-566; Robbins et al. (1995) Ann. N. Y. Acad. Sci. 752:492-505; Linn et al. (1995) Cire. Res. 76:584-591; Parmacek et al. (1994) Cell. Biol. 14:1870-1885; Hunter et al. (1993) Hypertension 22:608-617; and Sartorelli et al. (1992) Proc. Natl. Acad. Sci. USA 89:4047-4051.

Alternatively, certain advantages will be gained by positioning the coding nucleic acid segment under the control of a recombinant or heterologous promoter, which refers to a promoter that is not normally associated with a nucleic acid in its natural environment. A recombinant or heterologous enhancer refers also to an enhancer not normally associated with a nucleic acid sequence in its natural environment. Such promoters or enhancers often include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences are sometimes produced using recombinant cloning and/or nucleic acid amplification technology, including PCR, in connection with the compositions disclosed herein (see U.S. Pat. Nos. 4,683,202, 5,928,906, each incorporated herein by reference).

In some embodiments, the vectors of the disclosure include one or more polyA signals. Illustrative poly A signals useful in the vectors of the disclosure include the short polyA signal and the bGH polyA signal. In some embodiments, the vectors of the disclosure include one or more 3′ elements. Illustrative 3′ elements include the woodchuck hepatitis virus posttranscriptional regulatory element (WPRE).

### Gene Therapy Vector Compositions

To prepare the composition, the vectors and/or the cells are generated, and the vectors or cells are purified as necessary or desired. The vectors, and/or other agents are sometimes suspended in a pharmaceutically acceptable carrier. In some embodiments, the composition is lyophilized. These compounds and cells are often adjusted to an appropriate concentration, and optionally combined with other agents. The absolute weight of a given compound and/or other agent included in a unit dose varies widely. The dose and the number of administrations are contemplated to be optimized by those skilled in the art.

For example, in some embodiments, about 10²-10¹⁰ vector genomes (vg) are be administered. In some embodiments, the dose be at least about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes. In some embodiments, the dose be about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes.

Daily doses of the compounds vary as well. Such daily doses often range, for example, from at least about 10² vg/day, about 10³ vg/day, about 10⁴ vg/day, about 10⁵ vg/day, about 10⁶ vg/day, about 10⁷ vg/day, about 10⁸ vg/day, about 10⁹ vg/day, about 10¹⁰ vg/day, or more vector genomes per day.

In some embodiments, the method of the disclosure comprises administering a vector or vector system of the disclosure (e.g. an rAAV vector) by intracardiac injection, intramyocardiac injection, endocardial injection, intracardiac catheterization, or systemie administration. In some embodiments, the subject (e.g., a human) is treated by administering between about 1×10⁸ and about 1×10¹⁵ GC of a vector (e.g., an AAV vector or lentiviral vector) by intracardiac injection, intramyocardiac injection, endocardial injection, intracardiac catheterization, or systemic administration. In some embodiments, the subject is treated by administering between about 1×10⁸ and about 1×10¹⁵ GC, between about 1×10⁸ and about 1×10¹⁵ GC, between about 1×10⁹ and about 1×10¹⁴ GC, between about 1×10¹⁰ and about 1×10¹³ GC, between about 1×10¹¹ and about 1×10¹² GC, or between about 1×10¹² and about 1×10¹³ GC of vector. In some embodiments, the subject is treated by administering between about 1×10⁸ and about 1×10¹⁰ GC, between about 1×10⁹ and about 1×10¹¹ GC, between about 1×10¹⁶ and about 1×10¹² GC, between about 1×10¹¹ and about 1×10¹³ GC, between about 1×10¹² and about 1×10¹⁴ GC, or between about 1×10¹³ and about 1×10¹⁵ GC of vector. In some embodiments, the subject is treated by administering at least 1×10⁸, at least about 1×10⁹, at least about 1×10¹⁰, at least about 1×10¹¹, at least about 1×10¹², at least about 1×10¹³, or at least about 1×10¹⁵ GC of vector. In some embodiments, the subject is treated by administering at most 1×10⁸, at most about 1×10⁹, at most about 1×10¹⁰, at most about 1×10¹¹, at most about 1×10¹², at most about 1×10¹³, or at most about 1×10¹⁵ GC of vector. In some embodiments, the subject (e.g., a human) is treated by administering between about 1×10⁸ and about 1×10¹⁵ GC/kg of a vector (e.g., an AAV vector or lentiviral vector) by intracardiac injection or systemically. In some embodiments, the subject is treated by administering between about 1×10⁸ and about 1×10¹⁵ GC/kg, between about 1×10⁸ and about 1×10¹⁵ GC/kg, between about 1×10⁹ and about 1×10¹⁴ GC/kg, between about 133 10¹⁰ and about 1×10¹³ GC/kg, between about 1×10¹¹ and about 1×10¹² GC/kg, or between about 1×10¹² and about 1×10¹³ GC/kg of vector. In some embodiments, the subject is treated by administering between about 1×10⁸ and about 1×10¹⁰ GC/kg, between about 1×10⁹ and about 1×10¹¹ GC/kg, between about 1×10¹⁰ and about 1×10¹² GC/kg, between about 1×10¹¹ and about 1×10¹³ GC/kg, between about 1×10¹² and about 1×10¹⁴ GC kg, or between about 1×10¹³ and about 1×10¹⁵ GC/kg of vector. In some embodiments, the subject is treated by administering at least 1×10⁸, at least about 1×10⁹, at least about 1×10¹⁰, at least about 1×10¹¹, at least about 1×10¹², at least about 1×10¹³, or at least about 1×10¹⁵ GC/kg of vector. In some embodiments, the subject is treated by administering at most 1×10⁸, at most about 1×10⁹, at most about 1×10¹⁰, at most about 1×10¹¹, at most about 1×10¹², at most about 1×10¹³, or at most about 1×10¹⁵ GC/kg of vector. It will be appreciated that the amount of vectors and for use in treatment will vary not only with the particular carrier selected but also with the route of administration, the nature of the condition being treated and the age and condition of the patient. Ultimately, in some embodiments, the attendant health care provider will determine proper dosage. A pharmaceutical composition is contemplated to be formulated with the appropriate ratio of cach compound in a single unit dosage form for administration.

The compositions are sometimes formulated for sustained release (for example, using microencapsulation, see WO 94/07529, and/or U.S. Pat. No. 4,962,091). The formulations, where appropriate, are conveniently presented in discrete unit dosage forms and, in some embodiments, are prepared by any of the methods well known to the pharmaceutical arts. Such methods often include the step of mixing the therapeutic agent with liquid carriers, solid matrices, semi-solid carriers, finely divided solid carriers or combinations thereof, and then, if necessary, introducing or shaping the product into the desired delivery system.

One or more suitable unit dosage forms containing the compounds, in some embodiments, are administered by a variety of routes including parenteral (including subcutaneous, intravenous, intramuscular and intraperitoneal), intracardially, pericardially, oral, rectal, dennal, transdermal, intrathoracie, intrapulmonary, and intranasal (respiratory) routes.

The gene therapy vectors provided herein are prepared in many forms that include aqueous solutions, suspensions, tablets, hard or soft gelatin capsules, and liposomes and other slow-release formulations, such as shaped polymeric gels. Administration of gene therapy vectors often involves parenteral or local administration in an aqueous solution. Similarly. compositions containing gene therapy vectors are sometimes administered in a device, scaffold, or as a sustained release formulation. Different types of formulating procedures are described in U.S. Pat. No. 6,306,434 and in the references contained therein.

Vectors, in some embodiments, are formulated for parenteral administration (e.g., by injection, for example, bolus injection or continuous infusion) and are often presented in unit dosage form in ampoules, prefilled syringes, small volume infusion containers or multi-dose containers with an added preservative. The pharmaceutical compositions often take the form of suspensions, solutions, or emulsions in oily or aqueous vehicles, and sometimes contain formulatory agents such as suspending, stabilizing and/or dispersing agents. Suitable carriers include saline solution, phosphate buffered saline, and other materials commonly used in the art.

The compositions sometimes also contain other ingredients such as agents useful for treatment of cardiac diseases, conditions and injuries, such as, for example, an anticoagulant (e.g., dalteparin (fragmin), danaparoid (orgaran), enoxaparin (lovenox), heparin, tinzaparin (innohep), and/or warfarin (coumadin), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univase), Perindopril (Aceon), Quinapril (Accupril), Ramipril (Altace), and/or Trandolapril (Mavik)), angiotensin Il receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diureties (e.g., Amiloride (Midamor), Bumetanide (Burnex), Chlorothiazide (Diuril), Chlorthalidone (Hygroton), Furosemide (Lasix), Hydro-chlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, Lanoxin, or any combination thereof.

Additional agents are sometimes included such as antibacterial agents, antimicrobial agents, anti-viral agents, biological response modifiers, growth factors; imnune modulators, monoclonal antibodies and/or preservatives. The compositions provided herein are contemplated to also be used in conjunction with other forms of therapy.

The viral vectors described herein are suitable for administration to a subject to treat a disease or disorder. In some embodiments, such a composition is in a single dose, in multiple doses, in a continuous or intermittent manner, depending, for example, upon the recipient's physiological condition, whether the purpose of the administration is in response to traumatie injury or for more sustained therapeutic purposes, and other factors known to skilled practitioners. The administration of the compounds and compositions of provided herein, in some embodiments, are administered continuously over a preselected period of time or alternatively are administered in a series of spaced doses. Both local and systemic administration is contemplated. In some embodiments, localized delivery of a viral or non-viral vector is achieved. In some embodiments, localized delivery of cells and/or vectors is used to generate a population of cells within the heart. In some embodiments, such a localized population operates as “pacemaker cells” for the heart.

### Definitions

As used herein, the term “cardiomyopathy” refers to any disease or dysfunction of the myocardium (heart masele) in which the heart is abnormally enlarged, thickened and/or stiffened. As a result, the heart muscle's ability to pump blood is usually weakened. The etiology of the disease or disorder is, in some cases, inflammatory, metabolic, toxic, infiltrative, fibroplastic, hematological, genetic, or unknown in origin. There are two general types of cardiomyopathies: ischemie (resulting from a lack of oxygen) and non-ischemic. In some cases, a cardiomyopathy is arrhythmogenic right ventricular cardiomyopathy (ARVC) or arrhythmogenic cardiomyopathy (ACM).

“Heart failure (HF) is a complex clinical syndrome that often result from any structural or functional cardiovascular disorder causing systemic perfusion inadequate to meet the body's metabolic demands without excessively increasing left ventricular filling pressures. It is characterized by specific symptoms, such as dyspnea and fatigue, and signs, such as fluid retention. As used herein, “chronic heart failure” or “congestive heart failure” or “CHF” refer, interchangeably, to an ongoing or persistent forms of heart failure. Common risk factors for CHF include old age, diabetes, high blood pressure and being overweight. CHF is broadly classified according to the systolic function of the left ventricle as HF with reduced or preserved ejection fraction (HFrEF and HFpEF). The term “heart failure” does not mean that the heart has stopped or is failing completely, but that it is weaker than is normal in a healthy person. In some cases, the condition is mild, causing symptoms that are noticeable when exercising, in others, the condition is more severe, causing symptoms that are, in some cases, life-threatening, even while at rest. The most common symptoms of chronic heart failure include shortness of breath, tiredness, swelling of the legs and ankles, chest pain and a cough. In some embodiments, the methods of the disclosure decrease, prevent, or ameliorate one or more symptoms of CHF (e.g., HFrEF) in a subject suffering from or at risk for CHF (e.g., HFrEF). In some embodiments, the disclosure provides methods of treating CHF and conditions that sometimes lead to CHF.

As used herein “acute heart failure” or “decompensated heart failure” refer, interchangeably, to a syndrome of the worsening of signs and symptoms reflecting an inability of the heart to pump blood at a rate commensurate to the needs of the body at normal filling pressure. AHF typically develops gradually over the course of days to weeks and then decompensates requiring urgent or emergent therapy due to the severity of these signs or symptoms. In some cases, AHF is the result of a primary disturbance in the systolic or diastolic function of the heart or of abnormal venous or arterial vasoconstriction, but generally represents an interaction of multiple factors, including volume overload. The majority of patients with AHF have decompensation of chronic heart failure (CHF) and consequently much of the discussion of the pathophysiology, presentation, and diagnosis of CHF is directly relevant to an understanding of AHF. In other cases, AHF results from an insult to the heart or an event that impairs heart function, such as an acute myocardial infarction, severe hypertension, damage to a heart valve, abnormal heart rhythms, inflammation or infection of the heart, toxins and medications. In some embodiments, the methods of the disclosure decrease, prevent, or ameliorate one or more symptoms of AHF in a subject suffering from or at risk for AHF. In some embodiments, the disclosure provides methods of treating AHF and conditions that sometimes lead to AHF. In some cases, AHF is the result of ischemia associated with myocardial infarction.

As used herein, the terms “subject” or “individual” refers to any animal, such as a domesticated animal, a zoo animal, or a human. In some cases, the “subject” or “individual” is a mammal like a dog, cat, horse, livestock, a zoo animal, or a human. Alternatively, or in combination, the subject or individual is a domesticated animal such as a bird, a pet, or a farm animal. Specific examples of “subjects” and “individuals” include, but are not limited to, individuals with a cardiac disease or disorder, and individuals with cardiac disorder-related characteristics or symptoms, such as arrhythmogenic right ventricular cardiomyopathy (ARVC) or arrhythmogenic cardiomyopathy (ACM).

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (2002) Cold Spring Harbor Laboratory Press; Sohail (2004) Gene Silencing by RNA Interference: Technology and Application (CRC Press); Sell (2013) Stem Cells Handbook.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%. or alternatively 10%, or alternatively 5%, or alternatively 2%. It is to be understood, although not always explicitly stated, that all numerical designations are preceded by the term “about”. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cardiomyocyte” includes a plurality of cardiomyocytes.

Also as used herein, “and/or” refers to and encompasses any and all possible combinations of one or more of the associated listed items, as well as the lack of combinations when interpreted in the alternative (“or”).

“Administration,” “administering” and the like, when used in connection with a gene therapy vector or composition thereof as provided herein refer both to direct administration, which, in some cases includes administration to non-cardiomyocytes in vitro, administration to non-cardiomyocytes in vivo, administration to a subject by a medical professional or by self-administration by the subject and or to indirect administration, which, in some cases, is the act of prescribing a composition comprising a gene therapy vector provided herein. When used herein in reference to a cell, it refers to introducing a composition to the cell. Typically, an effective amount is administered, which amount is often to be determined by one of skill in the art. Any suitable method of administration is contemplated to be used. In some cases, a gene therapy vector is administered to the cells by, for example, by addition of the gene therapy vector to the cell culture media or injection in vivo to the site of cardiac injury. In some cases, administration to a subject is achieved by, for example, intravascular injection, intramyocardial delivery, and the like.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium, or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stern or progenitor cells, cardiac conducting cells and cardiac pacemaking cells that constitute the cardiac muscle, blood vessels and cardiac cell supporting structure. In some cases, cardiac cells are derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac α-actinin. The term “cardiomyocyte” as used herein is an umbrella term comprising any cardiomyocyte subpopulation or cardiomyocyte subtype, e.g., atrial, ventricular and pacemaker cardiomyocytes.

The term “culture” or “cell culture” means the maintenance of cells in an artificial, in vitro environment. A “cell culture system” is used herein to refer to culture conditions in which a population of cells are grown as monolayers or in suspension. “Culture medium” is used herein to refer to a nutrient solution for the culturing, growth, or proliferation of cells. Culture medium is characterized, in some cases, by functional properties such as, but not limited to, the ability to maintain cells in a particular state (e.g., a pluripotent state, a quiescent state, etc.), or to mature cells, such as, in some embodiments, to promote the differentiation of progenitor cells into cells of a particular lineage (e.g., a cardiomyocyte).

As used herein, the term “expression” or “express” refers to the process by which nucleic acids or polynucleotides are transcribed into mRNA and/or the process by which the transcribed mRNA is subsequently being translated into peptides, polypeptides, or proteins. If the polynucleotide or nucleic acid is derived from genomic DNA, in some cases, expression includes splicing of the mRNA in a eukaryotic cell. In some cases, the expression level of a gene is determined by measuring the amount of mRNA or protein in a cell or tissue sample.

As used herein, an “expression cassette” is a DNA polynucleotide comprising one or more polynucleotides or nucleic acids encoding protein(s) or nucleic acid(s) that is configured to express the polynucleotide in a host cell. Typically, expression of the polynucleotide(s) is placed under the control of certain regulatory elements, including constitutive or inducible promoters, tissue-specific regulatory elements, and enhancers. Such polynucleotides are said to be “operably linked to” or “operatively linked to” the regulatory elements (e.g., a promoter).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergie response, or other problem or complication, commensurate with a reasonable benefit/risk ratio.

“Treatment,” “treating,” and “treat” are defined as acting upon a disease, disorder, or condition with an agent to reduce or ameliorate harmful or any other undesired effects of the disease, disorder, condition and/or their symptoms.

As used herein, the term “effective amount” and the like refers to an amount that is sufficient to induce a desired physiologie outcome (e.g., treatment of a disease). An effective amount is sometimes administered in one or more administrations, applications or dosages. Such delivery is dependent on a number of variables including the time period which the individual dosage unit is to be used, the bioavailability of the composition, the route of administration, etc. It is understood, however, that specific amounts of the compositions (e.g., gene therapy vectors) for any particular subject depends upon a variety of factors including the activity of the specific agent employed, the age, body weight, general health, sex, and diet of the subject, the time of administration, the rate of excretion, the composition combination, severity of the particular disease being treated and form of administration.

As used herein, the term “equivalents thereof” in reference to a polypeptide or nucleic acid sequence refers to a polypeptide or nucleic acid that differs from a reference polypeptide or nucleic acid sequence, but retains essential properties (e.g., biological activity). A typical variant of a polynucleotide differs in nucleotide sequence from another, reference polynucleotide. Changes in the nucleotide sequence of the variant, in some cases, alters the amino acid sequence of a polypeptide encoded by the reference polynucleotide. In some cases, nucleotide changes result in amino acid substitutions, deletions, additions, fusions and truncations in the polypeptide encoded by the reference sequence. Generally, differences are limited so that the sequences of the reference polypeptide and the variant are closely similar overall and, in many regions, identical.

As used herein, the term “nucleic acid” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), eDNA, recombinant polynucleotides, vectors, probes, and primers. As used herein, the word “polynucleotide” or “nucleic acid” preceded by a gene name (for example, “PKP2 nucleic acid”) refers to a polynucleotide sequence encoding the corresponding protein (for example, a “PK.P2 protein”).

The terms “polypeptide,” “peptide,” and “protein.” are used interchangeably herein and refer to a polymeric form of amino acids of any length, which sometimes include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues, immunologically tagged proteins, and the like. As used berein, the word “protein” preceded by a gene name (for example, “PKP2 protein”) refers to either the native protein or a functional variant thereof. A “native protein” is a protein encoded by a genomic copy of a gene of an organism, preferably the organism for which the vector is intended (e.g., a human, a rodent, a primate, or an animal of veterinary interest), in any of the gene's functional isoforms or functional allelic variations.

As used herein, a “functional variant” or “variant” of a protein is a variant with any number of amino acid substitutions, insertions, truncations, or internal deletions that retains the functional attributes of the protein, including, e.g., the protein's ability to induce, in combination with other factors, organization of desmosomes. In some cases, functional variants are identified computationally, such as variants baving only conservative substitutions, or experimentally using in vitro or in vivo assays.

As used herein, a “codon variant” of a polynucleotide sequence is polynucleotide sequence that encodes the same protein as a reference polynucleotide sequence having one or more synonymous codon substitutions. Selection of synonymous codons is within the skill of those in the art, the coding as the genetic code being known. In some cases, codon optimization is performed using a variety of computational tools (such the GENSMART™ Codon Optimization tool available at www.genscript.com). Generally, codon optimization is used to increase the expression of protein in a heterologous system, for instance when a human coding sequence is expressed in a bacterial system. The term “codon variant” is intended to encompass both sequences that are optimized in this manner and sequences that are optimized for other purposes, such as removal of CpG islands and or cryptic start sites.

The term “vector” refers to a macromolecule or complex of molecules comprising a polynucleotide or protein to be delivered to a host cell, either in vitro or in vivo. A vector is sometimes a modified RNA, a lipid nanoparticle (encapsulating either DNA or RNA), a transposon, an adeno-associated virus (AAV) vector, an adenovirus, a retrovirus, an integrating lentiviral vector (LVV), or a non-integrating LVV. Thus, as used herein “vectors” include naked polynucleotides used for transformation (e.g. plasmids) as well as any other composition used to deliver a polynucleotide to a cell, included vectors capable of transducing cells and vectors useful for transfection of cells.

As used herein, the term “viral vector” refers either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also cell components in addition to nucleic acid(s).

The term “genetic modification” refers to a permanent or transient genetie change induced in a cell following introduction of new nucleic acid (i.e., nucleic acid exogenous to the cell). Genetic change is often accomplished by incorporation of the new nucleic acid into the genome of the cardiac cell, or by transient or stable maintenance of the new nucleic acid as an extrachromosomal element. Where the cell is a eukaryotic cell, a permanent genetic change is often achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary. and are not intended as limitations on the scope of the claims. Changes therein and other uses which are encompassed within the spirit of the disclosure as defined by the scope of the claims will occur to those skilled in the art.

### Example 1

Treatment of DSP siRNA-Treated Cells with AAV-PKP2 Gene Therapy

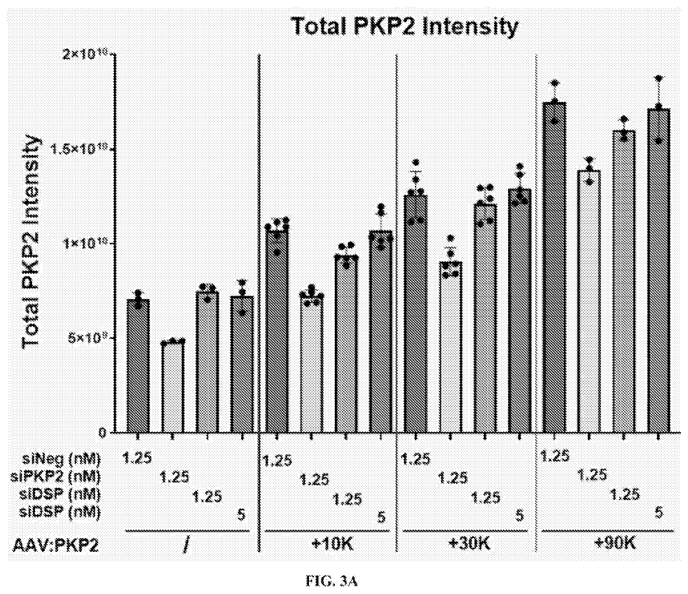

Induced pluripotent stem cell-derived cardiomyocytes (iPSCM) were treated with siRNA-PKP2, siRNA-DSP, or siRNA-control to reduce expression of the targeted gene. The cells were then treated with an AAV-PKP2 or left untreated to increase expression of PKP2. Cell viability was tested. In FIG. 2, total nuclear counts reflect the number of live cells. 20% cell death was observed with MOI was above 100 k. With up to 90 k MOI, no significant cell death was observed.

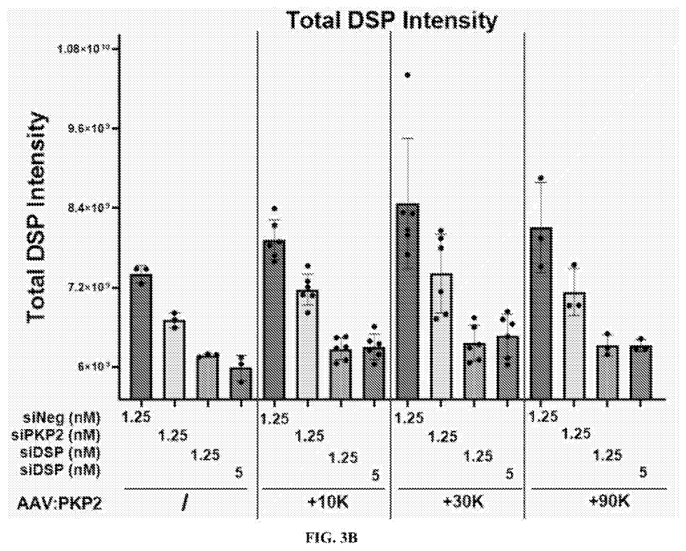

PKP2 and DSP immunofluorescence intensity was observed with the treated cells. DSP silencing by siRNA in iPSCM showed an overall reduction of total DSP protein level measured by fluorescence intensity at day 6 of silencing. FIG. 3A shows total PKP2 fluorescence intensity is reduced by siPKP2 and not by siDSP. There was a dose-dependent correlation of total PKP2 fluorescence intensity in response to increased MOIs of AAV:PKP2. In FIG. 3B it is shown that total DSP fluorescence intensity was reduced by both siDSP and siPKP2. In the presence of siDSP, total DSP fluorescence intensity did not show significant response to AAV:PKP2.

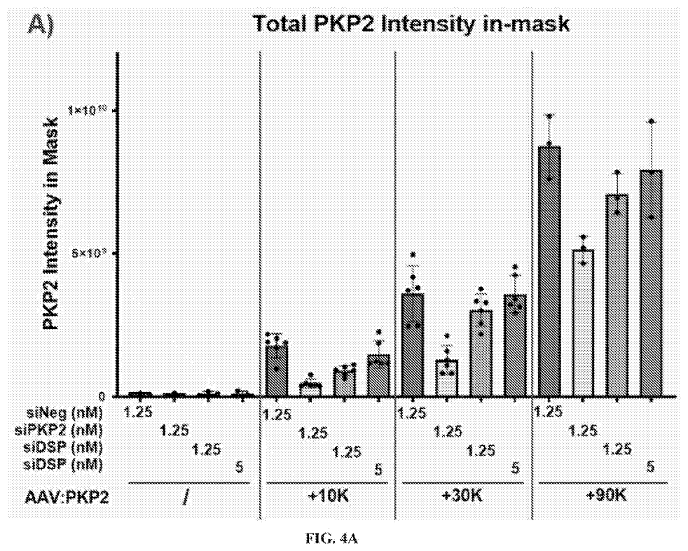

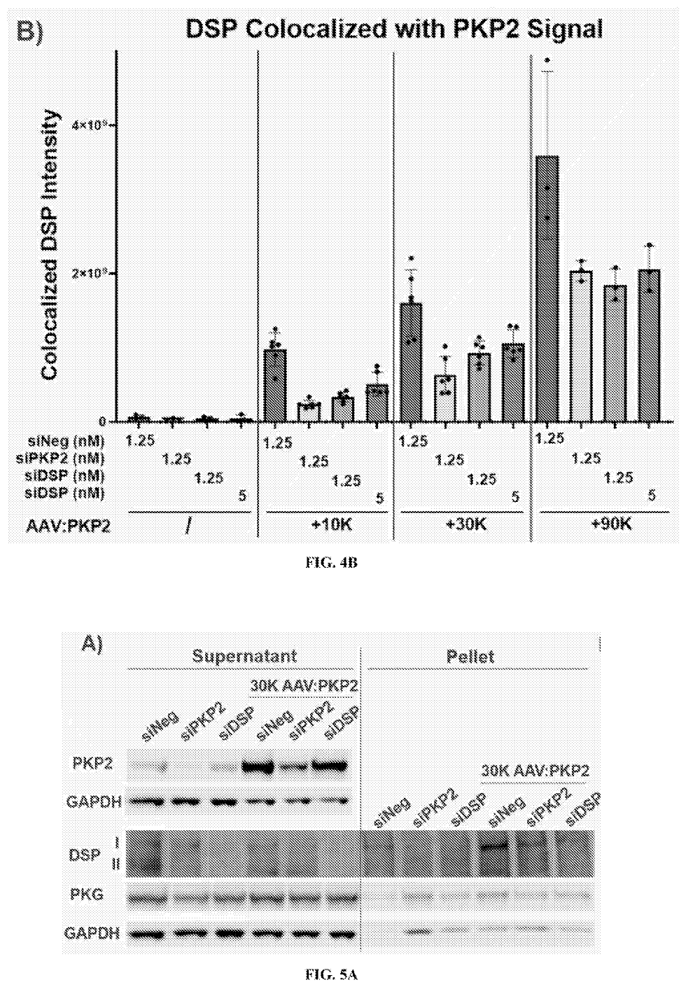

In contrast, increased PKP2 expression was found to stabilize DSP by colocalization in iPSCMs at day 4 of AAV transduction. FIG. 4A shows total PKP2 fluorescence intensity identified in mask, quantifying PKP2 colocalized with DSP, was significantly lower than the unmasked signal. A dose-dependent correlation of total masked PKP2 fluorescence intensity was found in response to increased MOIs of AAV:PKP2. FIG. 4B shows the fluorescence intensity of DSP in masked PKP2 measures the amount of DSP co-localized with PKP2. DSP co-localization with PKP2 was found to be enhanced with increased AAV:PKP2 MOI, suggesting DSP stabilization when co-localized with PKP2, as shown by the red box and the red arrow.

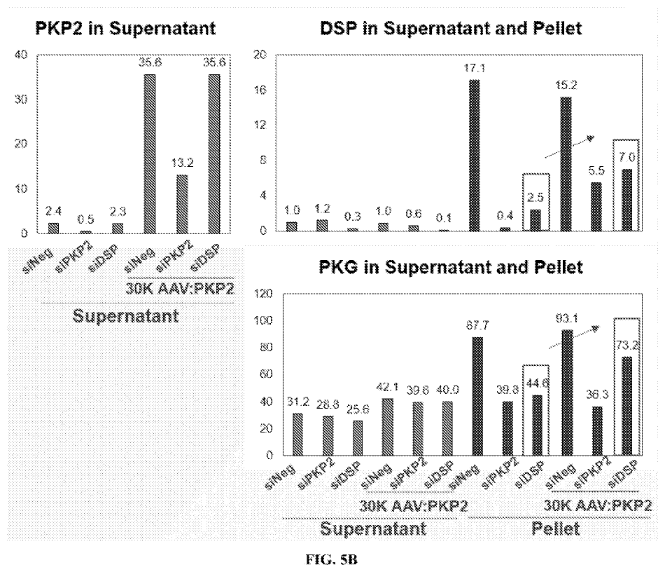

Increased expression of PKP2 was found to stabilize DSP in iPSCMs by semi-quantitative Western blot at day 4 after AAV transduction. FIG. 5A shows a semiquantitative Western blot illustrating expression of total PKP2 post silencing and AAV transduction in the supernatant. In addition, expression level of PKG and DSP were both detected in supernatant and pellet fractions. FIG. 5B shows quantification of protein levels in both fractions. There was about a 3-fold increase in pellet DSP observed in response to increased PKP2 expression by AAV:PKP2.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed. It is intended that the following claims define the scope of the disclosure and that methods and structures within the scope of these claims and their equivalents be covered thereby.

What is claimed is:

1. A method of treating a heart disease or disorder, the method comprising: (a) administering a first treatment comprising a viral vector comprising a nucleic acid encoding a plakophilin 2 (PKP2) polypeptide operatively linked to a promoter; and (b) administering a second treatment selected from the group consisting of a medication, a device, a procedure, and a lifestyle change.

2. The method of claim 1, wherein the medication comprises one or more of an antiarrhythmic, an angiotensin-converting enzyme (ACE) inhibitor, and a beta-blocker.

3. The method of claim 1 or claim 2, wherein the device comprises an implantable cardioverter-defibrillator (ICD).

4. The method of any one of claims 1 to 3, wherein the procedure comprises ablation.

5. The method of any one of claims 1 to 4, wherein the lifestyle change comprises one or more of diet, exercise, and stress reduction.

6. The method of any one of claims 1 to 5, wherein the viral vector is selected from the group consisting of an adeno-associated virus, an adenovirus, a lentivirus, a pox virus, a vaccinia virus, or a herpes virus.

7. The method of any one of claims 1 to 6, wherein the viral vector is an adeno-associated virus.

8. The method of claim 7, wherein the adeno-associated virus is selected from the group consisting of an AAV6, an AAV8, and an AAV9.

9. The method of claim 8, wherein the AAV9 is a variant of AAV9.

10. The method of any one of claims 1 to 9, wherein the heart disease or disorder is arrhythmogenic right ventricular cardiomyopathy (ARVC) or arrhythmogenic cardiomyopathy (ACM).

11. The method of any one of claims 1 to 10, wherein the promoter is a promoter that causes expression in tissues including the heart or a cardiac specific promoter.

12. The method of claim 11, wherein the cardiac specific promoter is a PKP2 promoter, a troponin promoter, or an alpha-myosin heavy chain promoter.

13. The method of any one of claims 1 to 12, wherein the viral vector comprises a 3′ element comprises a Woodchuck Hepatitis Virus Posttransciptional Regulatory Element (WPRE), a bovine growth hormone polyadenylation (bGH polyA) sequence, or a combination thereof.

14. The method of any one of claims 1 to 13, wherein the viral vector further comprises a cardiac specific enhancer.

15. The method of any one of claims 1 to 14, wherein the PKP2 polypeptide has an amino acid sequence of SEQ ID NO: 8.

16. The method of any one of claims 1 to 15, wherein the nucleic acid has a size less than or equal to about 4.7 kb.

17. The method of any one of claims 1 to 16, wherein the viral vector is administered in a pharmaceutically acceptable carrier or excipient comprising a buffer, a polymer, a salt, or a combination thereof.

18. The method of any one of claims 1 to 17, wherein the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain.

19. The method of any one of claims 1 to 18, wherein the method restores desmosome structure and/or function.

20. The method of any one of claims 1 to 19, wherein the method restores PKP2 mRNA expression and/or PKP2 protein and activity levels.

21. The method of any one of claims 1 to 20, wherein the method restores expression of one or more genes having a direct or indirect effect on one or more symptoms of the heart disease.

22. The method of claim 21, wherein the gene comprises one or more of Ryanodine Receptor 2 (Ryr2), Ankyrin-B (Ank2), Cacnalc (CaV1.2), triadin (Trdn), or calsequestrin-2 (Casq2).

23. The method of claim 1, wherein the individual is identified as having at least one variation in a desmosome protein.

24. The method of claim 22, wherein the desmosome protein is PKP2, desmoplakin (DSP), desmoglein (DSG2), desmocollin (DSC2), plakoglobin (JUP), a Connexin 43 (Cx43) gene, or transmembrane protein 43 (TMEM43).

25. The method of claim 23 or claim 24, wherein the variation comprises a deletion, an insertion, a single nucleotide variation, or a copy number variation.

26. The method of any one of claims 1 to 25, wherein the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14.

27. The method of any one of claims 1 to 26, wherein the method results in a reduction in dose or frequency of the second treatment.

28. The method of any one of claims 1 to 27, wherein the method results in increased exercise tolerance.

29. A method of treating a heart disease or disorder in a subject having a variation in a gene encoding a desmosome protein, the method comprising administering a first treatment comprising a viral vector comprising a nucleic acid encoding a plakophilin 2 (PKP2) polypeptide operatively linked to a promoter.

30. The method of claim 29, wherein the gene is a desmoplakin (DSP) gene, a desmoglein (DSG2) gene, a desmocollin (DSC2) gene, a plakoglobin (JUP) gene, a Connexin 43 (Cx43) gene, or a transmembrane protein 43 (TMEM43) gene.

31. The method of claim 29 or claim 30, wherein the variation comprises a deletion, an insertion, a single nucleotide variation, or a copy number variation.

32. The method of any one of claims 29 to 31, wherein the variation causes haploinsufficiency of DSP, DSG2, DSC2, JUP, Cx43, or TMEM43 protein.

33. The method of any one of claims 29 to 32, wherein the viral vector is selected from the group consisting of an adeno-associated virus, an adenovirus, a lentivirus, a pox virus, a vaccinia virus, or a herpes virus.

34. The method of any one of claims 29 to 33, wherein the viral vector is an adeno-associated virus.

35. The method of claim 34, wherein the adeno-associated virus is selected from the group consisting of an AAV6, an AAV8, and an AAV9.

36. The method of claim 35, wherein the AAV9 is a variant of AAV9.

37. The method of any one of claims 29 to 36, wherein the heart disease or disorder is arrhythmogenic right ventricular cardiomyopathy (ARVC) or arrhythmogenie cardiomyopathy (ACM).

38. The method of any one of claims 29 to 37, wherein the promoter is a promoter that causes expression in tissues including the heart or a cardiac specific promoter.

39. The method of claim 38, wherein the cardiac specific promoter is a PKP2 promoter, a troponin promoter, or an alpha-myosin heavy chain promoter.

40. The method of any one of claims 29 to 39, wherein the viral vector comprises a 3′ element comprises a Woodchuck Hepatitis Virus Posttransciptional Regulatory Element (WPRE), a bovine growth hormone polyadenylation (bGH polyA) sequence, or a combination thereof.

41. The method of any one of claims 29 to 40, wherein the viral vector further comprises a cardiac specific enhancer.

42. The method of any one of claims 29 to 41, wherein the PKP2 polypeptide bas an amino acid sequence of SEQ ID NO: 8.

43. The method of any one of claims 29 to 42, wherein the nucleic acid has a size less than or equal to about 4.7 kb.

44. The method of any one of claims 29 to 43, wherein the viral vector is administered in a pharmaceutically acceptable carrier or excipient comprising a buffer, a polymer, a salt, or a combination thereof.

45. The method of any one of claims 29 to 44, wherein the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure: left ventricular cardiomyopathy, dilation, or heart failure; atrial arrhythmias; syncope; palpitations; shortness of breath; or chest pain.

46. The method of any one of claims 29 to 45, wherein the method restores desmosome structure and/or function.

47. The method of any one of claims 29 to 46, wherein the method restores DSP, DSG2, DSC2, JUP, Cx43, or TMEM43 protein levels.

48. The method of any one of claims 29 to 47, wherein the method restores expression of one or more genes having a direct or indirect effect on one or more symptoms of the heart disease.

49. The method of claim 48, wherein the gene comprises one or more of Ryanodine Receptor 2 (Ryr2), Ankyrin-B (Ank2), Cacnale (CaV1.2), triadin (Trdn), or calsequestrin-2 (Casq2).

50. The method of any one of claims 29 to 49, wherein the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14.

51. The method of any one of claims 29 to 50, wherein the method results in a reduction in dose or frequency of a second treatment.

52. The method of claim 51, wherein the method further comprises administering a second treatment selected from the group consisting of a medication, a device, a procedure, and a lifestyle change.

53. The method of claim 52, wherein the medication comprises one or more of an antiarrhythmnic, an angiotensin-converting enzyme (ACE) inhibitor, and a beta-blocker.

54. The method of claim 52 or claim 53, wherein the device comprises an implantable cardioverter-defibrillator (ICD).

55. The method of any one of claims 52 to 54, wherein the procedure comprises ablation.

56. The method of any one of claims 52 to 55, wherein the lifestyle change comprises one or more of diet, exercise, and stress reduction.

57. The method of any one of claims 30 to 56, wherein the method results in increased exercise tolerance.

58. A method of restoring expression of one or more genes selected from Ryanodine Receptor 2 (Ryr2), Ankyrin-B (Ank2), Caenalc (CaV1.2), Triadin (Trdn), or Calsequestrin-2 (Casq2) in a subject in need thereof, comprising administering to the subject a viral vector comprising a nucleic acid encoding a plakophilin 2 (PKP2) polypeptide operatively linked to a promoter.

59. The method of claim 58, wherein the subject has a variation in a PKP2 gene, a desmoplakin (DSP) gene, a desmoglein (DSG2) gene, a desmocollin (DSC2) gene, a plakoglobin (JUP) gene, a Connexin 43 (Cx43) gene, or a transmembrane protein 43 (TMEM43) gene.

60. The method of claim 59, wherein the variation comprises a deletion, an insertion, a single nucleotide variation, or a copy number variation,

61. The method of claim 59 or claim 60, wherein the variation causes haploinsufficiency of DSP, DSG2, DSC2, JUP, Cx43, or TMEM43 protein.

62. The method of any one of claims 58 to 61, wherein the method restores DSP, DSG2, DSC2, JUP, Cx43, or TMEM43 protein levels.

63. The method of any one of claims 58 to 62, wherein the viral vector is selected from the group consisting of an adeno-associated virus, an adenovirus, a lentivirus, a pox virus, a vaccinia virus, or a herpes virus.

64. The method of any one of claims 58 to 63, wherein the viral vector is an adeno-associated virus.

65. The method of claim 64, wherein the adeno-associated virus is selected from the group consisting of an AAV6, an AAV8, and an AAV9.

66. The method of claim 65, wherein the AAV9 is a variant of AAV9 selected from the group consisting of CR9-10.

67. The method of any one of claims 58 to 66, wherein the subject has a heart disease or disorder comprising arrhythmogenic right ventricular cardiomyopathy (ARVC) or arrhythmogenic cardiomyopathy (ACM).

68. The method of any one of claims 58 to 67, wherein the promoter is a promoter that causes expression in tissues including the heart or a cardiac specific promoter.

69. The method of claim 68, wherein the cardiac specific promoter is a PKP2 promoter, a troponin promoter, or an alpha-myosin heavy chain promoter.

70. The method of any one of claims 58 to 59, wherein the viral vector comprises a 3′ element comprises a Woodchuck Hepatitis Virus Posttransciptional Regulatory Element (WPRE), a bovine growth hormone polyadenylation (bGH polyA) sequence, or a combination thereof.

71. The method of any one of claims 58 to 70, wherein the viral vector further comprises a cardiac specific enhancer.

72. The method of any one of claims 58 to 71, wherein the PKP2 polypeptide has an amino acid sequence of SEQ ID NO: 8.

73. The method of any one of claims 58 to 72, wherein the nucleic acid has a size less than or equal to about 4.7 kb.

74. The method of any one of claims 58 to 73, wherein the viral vector is administered in a pharmaceutically acceptable carrier or excipient comprising a buffer, a polymer, a salt, or a combination thereof.

75. The method of any one of claims 58 to 74, wherein the method reverses, reduces, or prevents at least one of fibrofatty tissue replacement; myocardial atrophy; ventricular dilation; ventricular arrhythmias; sudden cardiac death; exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain.

76. The method of any one of claims 58 to 75, wherein the method restores desmosome structure and/or function.

77. The method of any one of claims 58 to 76, whereis the viral vector is administered at a dose of about 1×10\hat{ }12, about 5×10\hat{ }12, about 1×10\hat{ }13, about 5×10\hat{ }13, about 1×10\hat{ }14, or about 5×10\hat{ }14.

78. The method of any one of claims 58 to 77, wherein the method results in a reduction in done or frequency of a second treatment.

79. The method of any one of claims 58 to 78, whereis the method results in increased exercise tolerance in the mobject.
